SEQUENCE LISTING (22 sequences)

SEQ ID NO: 1 - DNA, 21 bp, Artificial Sequence
  short hTERT FW
tgggttttagagttgattttt

SEQ ID NO: 2 - DNA, 20 bp, Artificial Sequence
  short hTERT RV
accacattaaacaatcccct

SEQ ID NO: 3 - DNA, 28 bp, Artificial Sequence
  short hTERT Probe
aattaggtataggggatttggttttagt

SEQ ID NO: 4 - DNA, 20 bp, Artificial Sequence
  long hTERT FW
agggttttagttagaagatt

SEQ ID NO: 5 - DNA, 24 bp, Artificial Sequence
  long hTERT RV
cattatatattaacttattcccac

SEQ ID NO: 6 - DNA, 27 bp, Artificial Sequence
  long hTERT probe
gtgttggggttatttttttgtatttgg

SEQ ID NO: 7 - DNA, 23 bp, Artificial Sequence
  Line-1 converted FW
ttttgagttaggtgtgggatata

SEQ ID NO: 8 - DNA, 21 bp, Artificial Sequence
  Line-1 converted RV
aaaatcaaaaaattccctttc

SEQ ID NO: 9 - DNA, 26 bp, Artificial Sequence
  Line-1 converted probe
tgggagtgatttgattttttaggtgy

SEQ ID NO: 10 - DNA, 20 bp, Artificial Sequence
  Line-1 genomic FW
gagccaggtgtgggatataa

SEQ ID NO: 11 - DNA, 20 bp, Artificial Sequence
  Line-1 genomic RV
tcaaagaaaggggtgacaga

SEQ ID NO: 12 - DNA, 25 bp, Artificial Sequence
  Line-1 genomic probe
tgggagtgacccgattttccaggtg

SEQ ID NO: 13 - DNA, 24 bp, Artificial Sequence
  IPC FW
ggtgatttttaataatttttgagg

SEQ ID NO: 14 - DNA, 21 bp, Artificial Sequence
  IPC RV
actttcaacccattacctcat

SEQ ID NO: 15 - DNA, 28 bp, Artificial Sequence
  IPC probe
agtgttcgtataggtttttttttttagt

SEQ ID NO: 16 - DNA, 85 bp, Artificial Sequence
  short hTERT gBlock
tgggttttagagttgatttttgtgaattaaattaaaattaggtataggggatttggttttagtataggggattgtttaatgtggt

SEQ ID NO: 17 - DNA, 235 bp, Artificial Sequence
  long hTERT gBlock
agggttttagttagaagattagggtttttttagtttttttgtatattcgagtttttggggggttttgtgatattttatgttttaaattaggatgtttgtagagggagttggtagtagatttcgttagaggtaatatagtttttgggttggggatttcgacgtggtgttggggttatttttttgtatttgggggagggttagggttttttttgtgggaataagttaatatataatg

SEQ ID NO: 18 - DNA, 148 bp, Artificial Sequence
  Line-1 converted gBlock
ttttgagttaggtgtgggatataattttttggtgtgttgttttttaagttttttggaaaagtgtagtatttagggtgggagtgatttgattttttaggtgttgtttgttatttttttttttgattaggaaagggaattttttgatttt

SEQ ID NO: 19 - DNA, 119 bp, Artificial Sequence
  Line-1 genomic gBlock
gagccaggtgtgggatataatctcctggtgtgctgttttttaagccctttggaaaagtgcagtatttagggtgggagtgacccgattttccaggtgccgtctgtcacccctttctttga

SEQ ID NO: 20 - DNA, 99 bp, Artificial Sequence
  IPC gBlock
ggtgatttttaataatttttgagggagtattggtataggcgatagagtgttcgtataggtttttttttttagtggatgatgaggtaatgggttgaaagt

SEQ ID NO: 21 - DNA, 25 bp, Artificial Sequence
  alternative probe Line-1 genomic
tgggagtgacccaattttccaggtg

SEQ ID NO: 22 - DNA, 26 bp, Artificial Sequence
  alternative probe Line-1 converted
tgggagtgatttaattttttaggtgy

(19) United States
(12) Patent Application Publication
(10) Pub. No.: US 2022/0372574 A1
(43) Pub. Date: Nov. 24, 2022

(54) METHOD FOR DETERMINING GLOBAL BISULFITE CONVERSION EFFICIENCY

(71) Applicant: Erasmus University Medical Center Rotterdam, Rotterdam (NL)

(72) Inventor: Athina VIDAKI, Rotterdam (NL)

(21) Appl. No.: 17/642,104

(22) PCT Filed: Sep. 11, 2020
(86) PCT No.: PCT/EP2020/075553

§ 371 Date: Mar. 10, 2022

(30) Foreign Application Priority Data
Sep. 11, 2019 (EP) 19386037.6

(51) Int. Cl. C12Q 1/6883

(52) U.S. Cl. CPC C12Q 1/6883; C12Q 1/6851

(57) ABSTRACT

The present invention relates to a method to determine bisulfite conversion of unmethylated cytosine to uracil in genomic DNA, comprising the steps of providing a first set of amplification primers for amplifying bisulfite converted copies of a repetitive DNA element by qPCR and a second set of amplification primers for amplifying unconverted copies of said repetitive DNA element by qPCR, performing a multiplex qPCR with said first and second set of amplification primers to generate amplicons, and determining the bisulfite conversion efficiency by comparing the amounts of said first and second amplicon.

### FIELD OF THE INVENTION

The invention is in the field of molecular biology, in particular to measurement of nucleic acid characteristics in biological samples. The invention relates to methods of studying chemical modification of nucleic acid during bisulfite conversion of unmethylated cytosine to uracil, and to kits-of-parts useful in such methods.

### BACKGROUND OF THE INVENTION

Epigenetics is the study of heritable modifications in gene expression that are not described by changes in DNA sequence, but rather by different DNA base modifications and histone alternations. DNA methylation is an epigenetic modification in which methyl groups are added covalently to the 5′ carbon of cytosine. Cytosine methylation in particular mainly appears in cytosine-guanine dinucleotides (5′-CpG-3′) in animals and is the most widely studied epigenetic marker, as it plays a significant role in inter alia gene regulation, aging, imprinting, X chromosome inactivation, disease development, cancer and silencing of repetitive DNA regions.

To study DNA methylation with molecular-based techniques, CpG methylation differences need to be translated to DNA sequence alternations. To achieve this, most techniques use the unique properties of sodium bisulfite (NaHSO₃). During bisulfite treatment the non-methylated cytosines are converted to uracils, whereas the methylated cytosines remain intact. Subsequent PCR amplification replaces uracils with thymines, allowing epigenetic modifications to be detectable as a C/T variation. This protocol was developed in 1992, and became the gold standard in DNA methylation studies as it can chemically differentiate between methylated and non-methylated cytosine bases.

Despite its great power, bisulfite treatment itself has some key disadvantages that affect downstream analysis. It uses harsh chemical conditions and high temperatures that cause DNA fragmentation and DNA loss. Several studies comparing different commercially available kits conclude that their performance depends on the treatment time and initial amount of DNA used in the procedure. Manufacturers of such kits mostly claim a performance of complete conversion of all unmethylated cytosines in the sample DNA (>99% conversion rate) with minimal DNA degradation (>80% DNA recovery). For researchers, there are currently no methods available to practically and simultaneously assess these numbers.

Naturally, lacking this information of converted-DNA quantity and quality can affect the accurate detection of DNA methylation due to bisulfite PCR amplification biases, which highly depend on the amount of bisulfite DNA used as template in the PCR reaction. Also, every conversion kit has a different protocol, mostly with respect to the incubation time and temperature of the bisulfite conversion reaction, which causes variable levels of conversion efficiency, of DNA recovery and of DNA fragmentation. The bisulfite conversion efficiency should be close to, if not at, 100%, so that false positive results are avoided. DNA recovery should also be accurately known, so that optimal PCR conditions are ensured. Lastly, DNA fragmentation should also be calculated to avoid false negative PCR results.

The problem of unknown DNA input in bisulfite PCR (i.e. the PCR reaction following the bisulfite conversion) and downstream analysis is extensive, as it can directly influence the detected DNA methylation levels, the reproducibility/robustness of bisulfite DNA-based assays as well as the final statistical evaluation of research outcomes that are based on the detected DNA methylation levels in health and disease studies and applications. It has been shown that the 95%-confidence interval of measured DNA methylation becomes wider with decreasing starting amounts of bisulfite DNA, which can be highly crucial when limited DNA amounts are used, such as for example in the common FFPE samples in medical fields and trace evidence in forensics.

As indicated, there is currently no accurate method to assess the quantity and quality of bisulfite-converted DNA. Although some methods exist to assess bisulfite-converted DNA quantity (e.g. Nanodrop spectrophotometer (ThermoFisher, USA), Qubit fluorometer (ThermoFisher) and Bioanalyzer (Agilent, USA) in RNA mode), none of this methods have been developed for bisulfite-converted DNA in particular but some researchers use the RNA mode of these instruments as the converted DNA is expected to be mostly single-stranded. In effect, no instrument/method exists so far to assess the conversion efficiency of a bisulfite DNA sample.

Thus far, no combined method to accurately and simultaneously assess both the quality and quantity of bisulfite converted DNA is available.

It is therefore an aim of the present invention to provide for methods and means that can be used to assess the quantity and/or quality of bisulfite-converted DNA. In addition, it is an aim of the present invention to provide for methods and means to assess bisulfite conversion efficiency of bisulfite-converted DNA, preferably based on genomic DNA samples.

### SUMMARY OF THE INVENTION

The present inventor has now discovered methods and means to assess the quality and/or quantity of bisulfite-converted DNA. More specifically, the inventor has discovered a method for determining a global (genome-wide) bisulfite conversion efficiency, by determining a bisulfite conversion status (i.e. bisulfite converted/unconverted ratio) of hundreds of copies of a repetitive DNA sequence, and by comparing the amount of bisulfite-converted and bisulfite-unconverted copies of said repetitive DNA sequence. Moreover, the inventor established methods for simultaneously determining a global bisulfite conversion efficiency, a bisulfite-converted DNA quantity, a level of DNA fragmentation following bisulfite conversion, and potential levels of PCR inhibition. These methods employ qPCR.

To that extent, the invention provides in a first aspect a method to determine bisulfite conversion efficiency following bisulfite conversion of unmethylated cytosine to uracil in a genomic DNA sample, the method comprising the steps of:

a) providing a sample of bisulfite-treated genomic DNA, said genomic DNA comprising a multi-copy target DNA sequence that is a repetitive DNA element, and wherein said bisulfite treatment converts unmethylated cytosine in the sequence of said repetitive DNA element to uracil to thereby generate bisulfite-converted copies of said repetitive DNA element;
b) providing a first set of amplification primers for amplifying by qPCR bisulfite-converted copies of said repetitive DNA element present in said bisulfite-treated genomic DNA sample and for generating a first amplicon, and providing a first detection probe labeled with a first detectable label for detecting said first amplicon by qPCR;
c) providing a second set of amplification primers for amplifying by qPCR unconverted copies of said repetitive DNA element present in said bisulfite-treated genomic DNA and for generating a second amplicon, and providing a second detection probe labeled with a second detectable label for detecting said second amplicon by qPCR;
d) performing a multiplex qPCR with said first and second set of amplification primers and probes and using said bisulfite-treated genomic DNA sample as a PCR template to generate said first amplicon with said first primer set from converted copies of said repetitive DNA element present in said bisulfite-treated genomic DNA sample, and to generate said second amplicon with said second primer set from unconverted copies of said repetitive DNA element present in said bisulfite-treated genomic DNA sample;
e) optionally, providing a first qPCR standard curve for determining the amount of converted copies of said repetitive DNA element present in said bisulfite-treated genomic DNA sample (as present prior to the multiplex qPCR of step d)), and providing a second qPCR standard curve for determining the amount of unconverted copies of said repetitive DNA element present in said bisulfite-treated genomic DNA sample (as present prior to the multiplex qPCR of step d)), wherein said first and second standard curves are obtained by performing a multiplex qPCR on a reference sample comprising as a PCR template a known amount of (i) a first synthetic DNA standard consisting of an oligonucleotide comprising a probe binding side complementary to said first detection probe flanked by primer binding sides complementary to said first set of amplification primers, preferably the oligonucleotide having a length (in bases) equal to said first amplicon (plus or minus 5-30 bases), preferably said primer binding sides being located at the 5′- and 3′-end of the oligonucleotide, preferably said first synthetic DNA standard consists of an oligonucleotide having the nucleotide sequence of said first amplicon, and (ii) a second synthetic DNA standard consisting of an oligonucleotide comprising a probe binding side complementary to said second detection probe flanked by primer binding sides complementary to said second set of amplification primers, preferably the oligonucleotide having a length (in bases) equal to said second amplicon (plus or minus 5-30 bases), preferably said primer binding sides being located at the 5′- and 3′-end of the oligonucleotide, preferably said second synthetic DNA standard consists of an oligonucleotide having the nucleotide sequence of said second amplicon, preferably wherein said first set of amplification primers and said first detection probe are used to amplify and quantify by qPCR said first synthetic DNA standard, and wherein said second set of amplification primers and said second detection probe are used to amplify and quantify by qPCR said second synthetic DNA standard;
f) determining the amount or concentration of converted and unconverted copies of said repetitive DNA element in said bisulfite-treated genomic DNA sample based on the generation of said first and second amplicon, respectively in step d), optionally based on said first and second qPCR standard curve provided in step e);
g) calculating the bisulfite conversion efficiency of the bisulfite treatment based on the ratio between the amount or concentrations of converted and unconverted copies of said repetitive DNA element present in said bisulfite-treated genomic DNA sample determined in step f).

In a preferred embodiment, the present invention provides a method to determine bisulfite conversion efficiency following bisulfite conversion of unmethylated cytosine to uracil in a genomic DNA sample, the method comprising the steps of:

a) providing a sample of bisulfite-treated genomic DNA, said genomic DNA comprising a multi-copy target DNA sequence that is a repetitive DNA element, and wherein said bisulfite treatment converts unmethylated cytosine in the sequence of said repetitive DNA element to uracil to thereby generate bisulfite-converted copies of said repetitive DNA element;
b) providing a first set of amplification primers for amplifying by qPCR bisulfite-converted copies of said repetitive DNA element present in said bisulfite-treated genomic DNA sample and for generating a first amplicon, and providing a first detection probe labeled with a first detectable label for detecting said first amplicon by qPCR;
c) providing a second set of amplification primers for amplifying by qPCR unconverted copies of said repetitive DNA element present in said bisulfite-treated genomic DNA and for generating a second amplicon, and providing a second detection probe labeled with a second detectable label for detecting said second amplicon by qPCR;
d) performing a multiplex qPCR with said first and second set of amplification primers and probes and using said bisulfite-treated genomic DNA sample as a PCR template to generate said first amplicon with said first primer set from converted copies of said repetitive DNA element present in said bisulfite-treated genomic DNA sample, and to generate said second amplicon with said second primer set from unconverted copies of said repetitive DNA element present in said bisulfite-treated genomic DNA sample;
e) providing a first qPCR standard curve for determining the amount of converted copies of said repetitive DNA element present in said bisulfite-treated genomic DNA sample (as present prior to the multiplex qPCR of step d)), and providing a second qPCR standard curve for determining the amount of unconverted copies of said repetitive DNA element present in said bisulfite-treated genomic DNA sample (as present prior to the multiplex qPCR of step d)), wherein said first and second standard curves are obtained by performing a multiplex qPCR on a reference sample comprising as a PCR template a known amount of (i) a first synthetic DNA standard consisting of an oligonucleotide comprising a probe binding side complementary to said first detection probe flanked by primer binding sides complementary to said first set of amplification primers, preferably the oligonucleotide having a length (in bases) equal to said first amplicon (plus or minus 5-30 bases), preferably said primer binding sides being located at the 5′- and 3′-end of the oligonucleotide, preferably said first synthetic DNA standard consists of an oligonucleotide having the nucleotide sequence of said first amplicon, and (ii) a second synthetic DNA standard consisting of an oligonucleotide comprising a probe binding side complementary to said second detection probe flanked by primer binding sides complementary to said second set of amplification primers, preferably the oligonucleotide having a length (in bases) equal to said second amplicon (plus or minus 5-30 bases), preferably said primer binding sides being located at the 5′- and 3′-end of the oligonucleotide, preferably said second synthetic DNA standard consists of an oligonucleotide having the nucleotide sequence of said second amplicon, preferably wherein said first set of amplification primers and said first detection probe are used to amplify and quantify by qPCR said first synthetic DNA standard, and wherein said second set of amplification primers and said second detection probe are used to amplify and quantify by qPCR said second synthetic DNA standard;
f) determining the amount or concentration of converted and unconverted copies of said repetitive DNA element in said bisulfite-treated genomic DNA sample based on the generation of said first and second amplicon, respectively in step d), and based on said first and second qPCR standard curve provided in step e);
g) calculating the bisulfite conversion efficiency of the bisulfite treatment based on the ratio between the amount or concentrations of converted and unconverted copies of said repetitive DNA element present in said bisulfite-treated genomic DNA sample determined in step f).

In an alternative embodiment, the present invention provides a method to determine bisulfite conversion efficiency following bisulfite conversion of unmethylated cytosine to uracil in a genomic DNA sample, the method comprising the steps of:
providing a template genomic DNA sample that is treated with bisulfite to convert unmethylated cytosine to uracil, said DNA sample comprising a multi-copy target DNA sequence that is a repetitive DNA element;
providing a first set of amplification primers for amplifying bisulfite converted copies of said repetitive DNA element by qPCR and for generating a first amplicon; and providing a first detection probe labeled with a first detectable label for detecting said first amplicon by qPCR;
providing a second set of amplification primers for amplifying unconverted copies of said repetitive DNA element by qPCR and for generating a second amplicon; and providing a second detection probe labeled with a second detectable label for detecting said second amplicon by qPCR;
performing a multiplex qPCR with said first and second set of amplification primers and probes to thereby generate said first amplicon with said first primer set and, if unconverted copies are present, said second amplicon with said second primer set;
optionally, providing a first qPCR standard curve for determining the amount of converted copies of said repetitive DNA element present in said bisulfite-treated genomic DNA sample, or the amount or concentration of the initial template of said first amplicon (as present prior to the multiplex qPCR of step d)), and providing a second qPCR standard curve for determining the amount of unconverted copies of said repetitive DNA element present in said bisulfite-treated genomic DNA sample, or amount or concentration of the initial template of said second amplicon (as present prior to the multiplex qPCR of step d)), wherein said first and second standard curves are obtained by performing a multiplex qPCR on a reference sample comprising as a PCR template a known amount of (i) a first synthetic DNA standard consisting of an oligonucleotide comprising a probe binding side complementary to said first detection probe flanked by primer binding sides complementary to said first set of amplification primers, preferably the oligonucleotide having a length (in bases) equal to said first amplicon (plus or minus 5-30 bases), preferably said primer binding sides being located at the 5′- and 3′-end of the oligonucleotide, preferably said first synthetic DNA standard consists of an oligonucleotide having the nucleotide sequence of said first amplicon, and (ii) a second synthetic DNA standard consisting of an oligonucleotide comprising a probe binding side complementary to said second detection probe flanked by primer binding sides complementary to said second set of amplification primers, preferably the oligonucleotide having a length (in bases) equal to said second amplicon (plus or minus 5-30 bases), preferably said primer binding sides being located at the 5′- and 3′-end of the oligonucleotide, preferably said second synthetic DNA standard consists of an oligonucleotide having the nucleotide sequence of said second amplicon, preferably wherein said first set of amplification primers and said first detection probe are used to amplify and quantify by qPCR said first synthetic DNA standard, and wherein said second set of amplification primers and said second detection probe are used to amplify and quantify by qPCR said second synthetic DNA standard;
determining the amount or concentration of the initial template of said first amplicon and said second amplicon in said bisulfite-treated genomic DNA sample, optionally based on said first and second qPCR standard curve, preferably on the basis of qPCR Cq values, and calculate the bisulfite conversion efficiency of the bisulfite treatment by comparing said amounts of said first and second amplicon.

Preferably, in a method of the invention, the standard curves are prepared independently from the qPCR reaction performed on the bisulfite-treated genomic DNA sample, but preferably using qPCR reactants from a single batch (e.g. preferably using the same qPCR mastermix with all components for quantitative PCR except sample DNA and using the same primer and probe mixtures). Thus, in a preferred embodiment of the invention, the multiplex qPCR on the reference sample is preformed independently from the multiplex qPCR on the bisulfite-treated genomic DNA sample.

In a preferred embodiment, said repetitive DNA element is a long interspersed nuclear element (LINE), preferably an L1 repetitive element (LINE 1).

In another preferred embodiment, said first set of amplification primers comprises a forward primer comprising the nucleotide sequence of SEQ ID NO:7 and a reverse primer comprising the nucleotide sequence of SEQ ID NO:8; and optionally wherein the first detection probe comprises the nucleotide sequence of SEQ ID NO:9.

In yet another preferred embodiment, said second set of amplification primers comprises a forward primer comprising the nucleotide sequence of SEQ ID NO:10 and a reverse primer comprising the nucleotide sequence of SEQ ID NO:11; and optionally wherein the second detection probe comprises the nucleotide sequence of SEQ ID NO:12.

In yet another preferred embodiment, said first synthetic DNA standard is the sequence of SEQ ID NO:18 and said second synthetic DNA standard is the sequence of SEQ ID NO:19.

In yet another preferred embodiment, the bisulfite conversion efficiency is calculated using the formula: (amount or concentration of converted copies of said repetitive DNA element present in said bisulfite-treated genomic DNA sample)/(amount or concentration of converted copies of said repetitive DNA element present in said bisulfite-treated genomic DNA sample+amount or concentration of unconverted copies of said repetitive DNA element present in said bisulfite-treated genomic DNA sample).

In yet another preferred embodiment, the first detectable label is TEX616 and/or the second detectable label is Cy5.

In yet another preferred embodiment, said method is a method for simultaneously determining a bisulfite conversion efficiency, a bisulfite converted DNA quantity and a level of DNA fragmentation following bisulfite conversion; wherein said bisulfite-treated genomic DNA sample further comprises a single copy gene sequence, said method further comprising the steps of:

h) providing a third set of amplification primers for amplifying by qPCR at least a part of said single-copy gene sequence that is bisulfite-converted and for generating a third amplicon; and a third detection probe labeled with a third detectable label for detecting said third amplicon by qPCR;
i) providing a fourth set of amplification primers for amplifying by qPCR at least a part of said single-copy gene sequence that is bisulfite-converted and for generating a fourth amplicon and a fourth detection probe labeled with a fourth detectable label for detecting said fourth amplicon by qPCR, wherein the third and fourth set of amplification primers are designed such that the length (in bp) of said fourth amplicon is longer than the length (in bp) of said third amplicon, preferably wherein the length of the third amplicon is about 30-100 bp, and preferably wherein the length of the fourth amplicon is between 150-500 bp;
j) performing the multiplex qPCR of step d) which further includes said third and fourth set of primers and probes to thereby further generate said third amplicon with said third primer set and said fourth amplicon with said fourth primer set;
k) optionally, providing a third qPCR standard curve for determining the amount of converted and optionally fragmented copies of said single copy gene sequence present in said bisulfite-treated genomic DNA sample (as present prior to the multiplex qPCR of step d)), and providing a fourth qPCR standard curve for determining the amount of converted and non-fragmented copies of said single-copy gene sequence present in said bisulfite-treated genomic DNA sample (as present prior to the multiplex qPCR of step d)), wherein said third and fourth standard curves are obtained by performing a multiplex qPCR on a reference sample comprising as a PCR template a known amount of (iii) a third synthetic DNA standard consisting of an oligonucleotide comprising a probe binding side complementary to said third detection probe flanked by primer binding sides complementary to said third set of amplification primers, preferably the oligonucleotide having a length (in bases) equal to said third amplicon (plus or minus 5-30 bases), preferably said primer binding sides being located at the 5′- and 3′-end of the oligonucleotide, preferably said third synthetic DNA standard consists of an oligonucleotide having the nucleotide sequence of said third amplicon, and (iv) a fourth synthetic DNA standard consisting of an oligonucleotide comprising a probe binding side complementary to said fourth detection probe flanked by primer binding sides complementary to said fourth set of amplification primers, preferably the oligonucleotide having a length (in bases) equal to said fourth amplicon (plus or minus 5-30 bases), preferably said primer binding sides being located at the 5′- and 3′-end of the oligonucleotide, preferably said fourth synthetic DNA standard consists of an oligonucleotide having the nucleotide sequence of said fourth amplicon,
preferably wherein said third set of amplification primers and said third detection probe are used to amplify and quantify by qPCR said third synthetic DNA standard, and wherein said fourth set of amplification primers and said fourth detection probe are used to amplify and quantify by qPCR said fourth synthetic DNA standard;
l) determining the amount or concentration of converted and optionally fragmented copies of said single copy gene sequence present in said bisulfite-treated genomic DNA sample based on the generation of said third amplicon, preferably on the basis of qPCR Cq values, to thereby provide a bisulfite-converted DNA quantity in said bisulfite-treated genomic DNA sample,
m) determining the amount or concentration of converted and non-fragmented copies of said single copy gene sequence present in said bisulfite-treated genomic DNA sample based on the generation of said fourth amplicon, preferably on the basis of qPCR Cq values, and provide the level of DNA fragmentation following bisulfite conversion by comparing the determined amount of said converted and optionally fragmented copies with the amount of converted and non-fragmented copies present in said bisulfite-treated genomic DNA sample;
n) optionally wherein the step of determining the amount or concentration of converted and optionally fragmented copies of said single copy gene sequence present in said bisulfite-treated genomic DNA sample and the step of determining the amount or concentration of converted and non-fragmented copies of said single copy gene sequence present in said bisulfite-treated genomic DNA sample in said bisulfite-treated genomic DNA sample is based on said third and fourth qPCR standard curve.

In yet another preferred embodiment, the present invention provides a method as described above, wherein said method is a method for simultaneously determining a bisulfite conversion efficiency, a bisulfite converted DNA quantity and a level of DNA fragmentation following bisulfite conversion; wherein said bisulfite-treated genomic DNA sample further comprises a single-copy gene sequence, said method further comprising the steps of:

h) providing a third set of amplification primers for amplifying by qPCR at least a part of said single-copy gene sequence that is bisulfite-converted and for generating a third amplicon; and a third detection probe labeled with a third detectable label for detecting said third amplicon by qPCR;
i) providing a fourth set of amplification primers for amplifying by qPCR at least a part of said single-copy gene sequence that is bisulfite-converted and for generating a fourth amplicon, and providing a fourth detection probe labeled with a fourth detectable label for detecting said fourth amplicon by qPCR, wherein the third and fourth set of amplification primers are designed such that the length (in bp) of said fourth amplicon is longer than the length (in bp) of said third amplicon;
j) performing the multiplex qPCR of step d) which further includes said third and fourth set of primers and probes to thereby further generate said third amplicon with said third primer set and said fourth amplicon with said fourth primer set;
k) providing a third qPCR standard curve for determining the amount of converted and optionally fragmented copies of said single copy gene sequence present in said bisulfite-treated genomic DNA sample, and providing a fourth qPCR standard curve for determining the amount of converted and non-fragmented copies of said single-copy gene sequence present in said bisulfite-treated genomic DNA sample, wherein said third and fourth standard curves are obtained by performing a multiplex qPCR on a reference sample comprising as a PCR template a known amount of (iii) a third synthetic DNA standard consisting of an oligonucleotide comprising a probe binding side complementary to said third detection probe flanked by primer binding sides complementary to said third set of amplification primers, and (iv) a fourth synthetic DNA standard consisting of an oligonucleotide comprising a probe binding side complementary to said fourth detection probe flanked by primer binding sides complementary to said fourth set of amplification primers;
l) determining the amount or concentration of converted and optionally fragmented copies of said single copy gene sequence present in said bisulfite-treated genomic DNA sample based on the generation of said third amplicon in step j), and based on said third qPCR standard curve provided in step k), to thereby provide a bisulfite-converted DNA quantity in said bisulfite-treated genomic DNA sample,
m) determining the amount or concentration of converted and non-fragmented copies of said single copy gene sequence present in said bisulfite-treated genomic DNA sample based on the generation of said fourth amplicon in step j), and based on said fourth qPCR standard curve provided in step k);
n) and calculate the level of DNA fragmentation following bisulfite conversion by comparing the amount of said converted and optionally fragmented copies with the amount of converted and non-fragmented copies present in said bisulfite-treated genomic DNA sample as determined in steps 1) and m).

In all aspects of this invention, PCR endpoints may be based on Cq values or on RFU following a defined number of cycles.

In yet another preferred embodiment, the third amplicon is 60-100 bps, preferably about 85 bps, and said fourth amplicon is 150-350 bps, preferably about 235 bps.

In yet another preferred embodiment, said single-copy gene sequence is a (human) telomerase reverse transcriptase gene (hTERT).

In yet another preferred embodiment, said third set of amplification primers comprises a forward primer comprising the nucleotide sequence of SEQ ID NO:1 and a reverse primer comprising the nucleotide sequence of SEQ ID NO:2; and optionally wherein the third detection probe comprises the nucleotide sequence of SEQ ID NO:3.

In yet another preferred embodiment, said fourth set of amplification primers comprises a forward primer comprising the nucleotide sequence of SEQ ID NO:4 and a reverse primer comprising the nucleotide sequence of SEQ ID NO:5; and optionally wherein the fourth detection probe comprises the nucleotide sequence of SEQ ID NO:6.

In yet another preferred embodiment, said third synthetic DNA standard is the sequence of SEQ ID NO:16 and said fourth synthetic DNA standard is the sequence of SEQ ID NO:17.

In yet another preferred embodiment, the amount of said first and second synthetic DNA standard relative to the amount of said third and fourth synthetic DNA standard in said reference sample reflect the ratio of copy numbers of said repetitive DNA element and said single copy gene sequence in genomic DNA in said bisulfite-treated genomic DNA sample, preferably wherein the ratio between said first and second synthetic DNA standard is about 1 and wherein the ratio between said third and fourth synthetic DNA standard is about 1, more preferably wherein the ratio between the copy number of said first, second third and fourth synthetic DNA standard in said reference sample is 200:200:1:1, respectively.

In another aspect, the present invention provides a method for simultaneously determining a bisulfite conversion efficiency, a bisulfite converted DNA quantity, a level of DNA fragmentation following bisulfite conversion, and PCR inhibition, said method comprising the steps of:
performing any of the methods as described herein above, and further comprising the steps of:
adding to said bisulfite-treated genomic DNA sample an artificial (synthetic) DNA sequence in a known amount, preferably wherein the artificial DNA sequence comprises the nucleotide sequence of SEQ ID NO:20;
providing a fifth set of amplification primers for amplifying by qPCR said artificial DNA sequence and for generating a fifth amplicon; and a fifth detection probe labeled with a fifth detectable label for detecting said fifth amplicon by qPCR;
performing said multiplex qPCR which further includes said fifth set of primers and probe to thereby further generate said fifth amplicon with said fifth primer set;
determining the amount of said fifth amplicon, preferably on the basis of qPCR Cq values, and determine the level of PCR inhibition by comparing the determined amount of said fifth amplicon in the said template bisulfite-converted DNA with the correspondent determined amount of said fifth amplicon in the DNA quantification standards, where inhibition is not expected.

In yet another preferred embodiment, said fifth set of amplification primers comprises a forward primer comprising the nucleotide sequence of SEQ ID NO:13 and a reverse primer comprising the nucleotide sequence of SEQ ID NO:14; and optionally wherein the fifth detection probe comprises the nucleotide sequence of SEQ ID NO:15.

In another aspect, the present invention provides a qPCR kit comprising
a first set of amplification primers for amplifying bisulfite converted copies of a genomic multi-copy target DNA sequence that is a repetitive DNA element and for generating a first amplicon;
a first detection probe labeled with a first detectable label for detecting said first amplicon;
a second set of amplification primers for amplifying unconverted copies of said repetitive DNA element by qPCR and for generating a second amplicon;
a second detection probe labeled with a second detectable label for detecting said second amplicon;

wherein said qPCR kit optionally further comprises
a third set of amplification primers for amplifying a part of a genomic single-copy gene sequence that is bisulfite converted and for generating a third amplicon;
a third detection probe labeled with a third detectable label for detecting said third amplicon;
a fourth set of amplification primers for amplifying a part of said genomic single-copy gene sequence that is bisulfite converted and for generating a fourth amplicon that is longer in length than said third amplicon;
a fourth detection probe labeled with a fourth detectable label for detecting said fourth amplicon; and/or
a fifth set of amplification primers for amplifying an internal positive control DNA sequence and for generating a fifth amplicon, preferably wherein said fifth set of amplification primers comprises a forward primer comprising the nucleotide sequence of SEQ ID NO:13 and a reverse primer comprising the nucleotide sequence of SEQ ID NO:14;
a fifth detection probe labeled with a fifth detectable label for detecting said fifth amplicon by qPCR, preferably wherein said fifth detection probe comprises the nucleotide sequence of SEQ ID NO:15;

wherein said qPCR kit optionally further comprises
a first synthetic DNA standard consisting of the nucleotide sequence of said first amplicon, preferably wherein said first synthetic DNA standard is the sequence of SEQ ID NO:18;
a second synthetic DNA standard consisting of the nucleotide sequence of said second amplicon, preferably wherein said second synthetic DNA standard is the sequence of SEQ ID NO:19; and/or;
a third synthetic DNA standard consisting of the nucleotide sequence of said third amplicon, preferably wherein said third synthetic DNA standard is the sequence of SEQ ID NO:16; and/or
a fourth synthetic DNA standard consisting of the nucleotide sequence of said fourth amplicon, preferably wherein said fourth synthetic DNA standard is the sequence of SEQ ID NO:17; and/or
an (artificial, non-human) internal positive control DNA sequence, preferably consisting of the sequence of SEQ ID NO:20.

In a preferred embodiment of a qPCR kit according to the invention, the kit comprises, in combination, the primers and probes of SEQ ID NO:7-9 adapted to support a 5plex PCR assay, preferably the primers and probes of SEQ ID NO:1-15 adapted to support a 5plex PCR assay and/or synthetic DNA standards of SEQ ID NOs:16-19, said kit optionally further comprising the internal positive control comprising the DNA sequence of SEQ ID NO:20.

In a preferred embodiment of a method of the invention, the first, second, third, fourth and fifth detectable label are different.

In another aspect, the invention provides a use of a first and second synthetic oligonucleotide, preferably an oligonucleotide of SEQ ID NO:18 or 19 as a first synthetic oligonucleotide and an oligonucleotide of SEQ ID NO:16 or 17 as a second synthetic oligonucleotide, as a DNA standard in a method for measuring bisulfite conversion efficiency in a bisulfite treated genomic DNA sample; wherein said first synthetic oligonucleotide has a nucleotide sequence that corresponds to the sequence of a bisulfite converted copy of a repetitive (genomic) DNA element and said second synthetic oligonucleotide has a nucleotide sequence that corresponds to the sequence of a non-bisulfite converted copy of said (genomic) repetitive DNA element.

In another aspect, the invention provides a nucleic acid, preferably a synthetic oligonucleotide, comprising or consisting a sequence of SEQ ID NOs:1-20.

### FIGURE LEGENDS

FIG. 1. qBiCo Method Development Using Synthetic DNA Standards (gBlocks) with Concentrations Ranging from 50-0.39 ng/μl

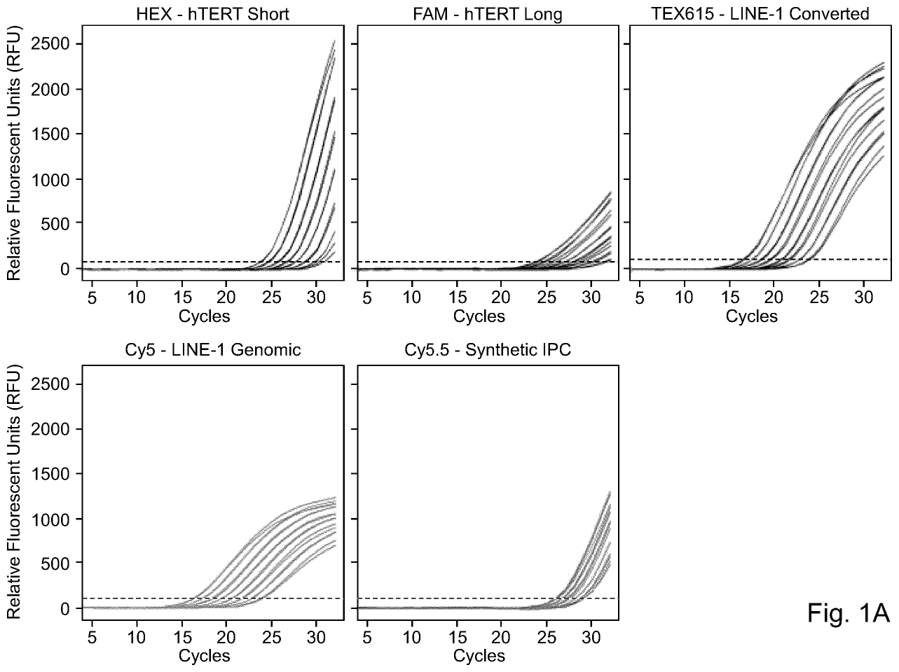

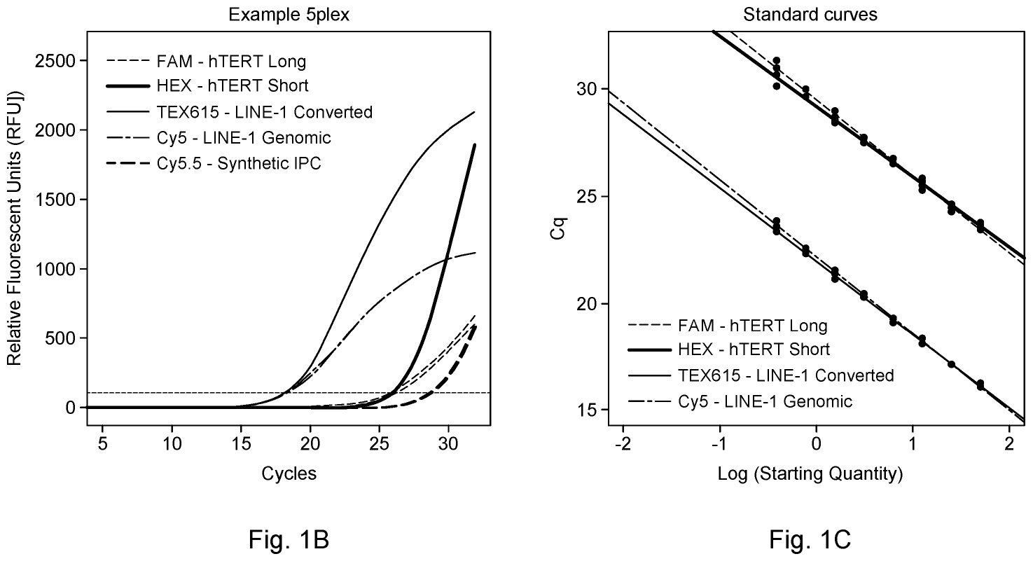

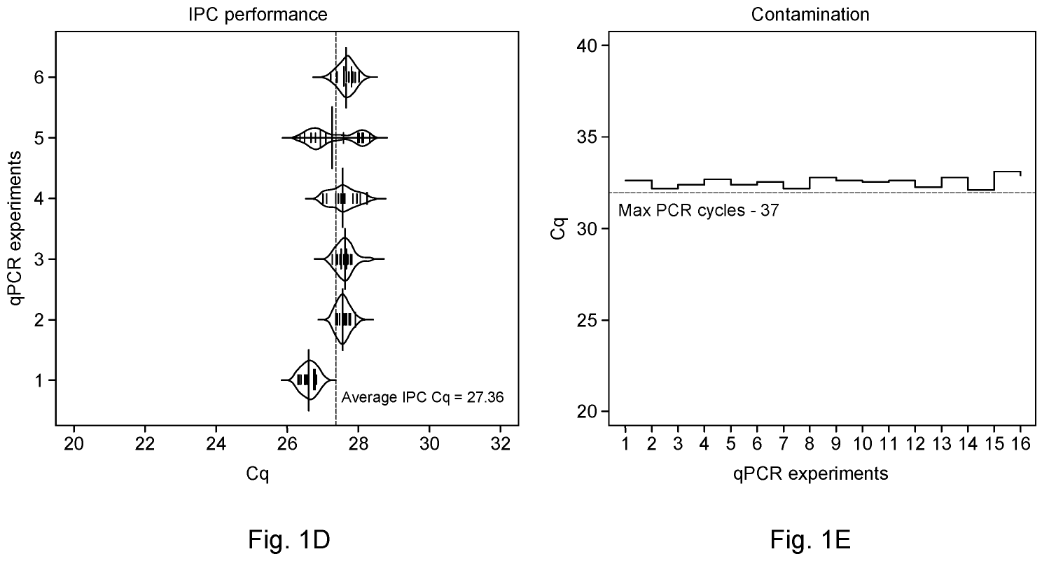

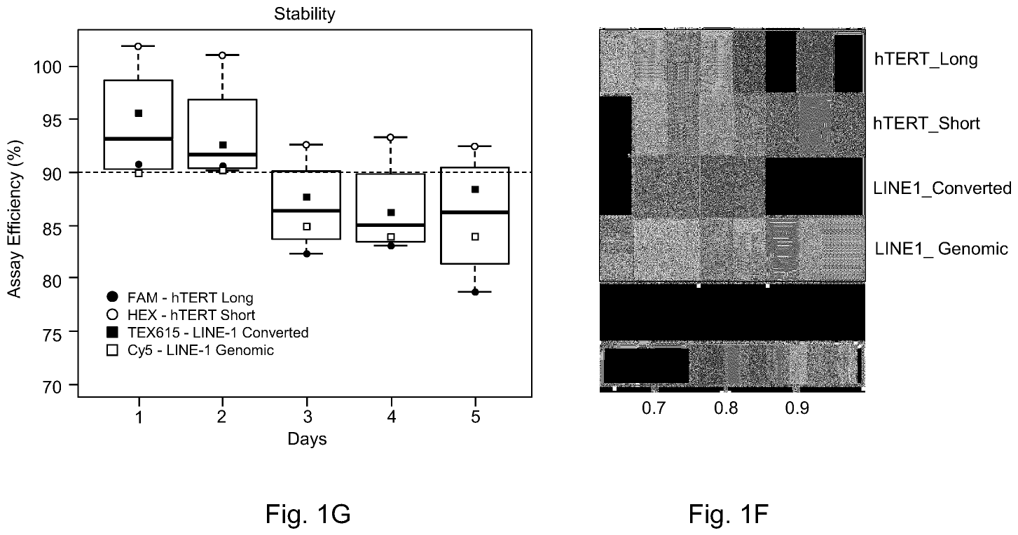

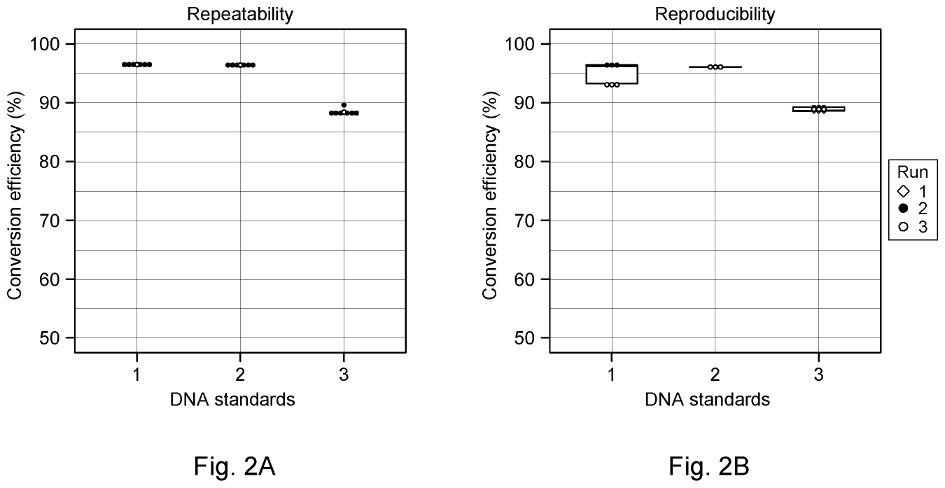

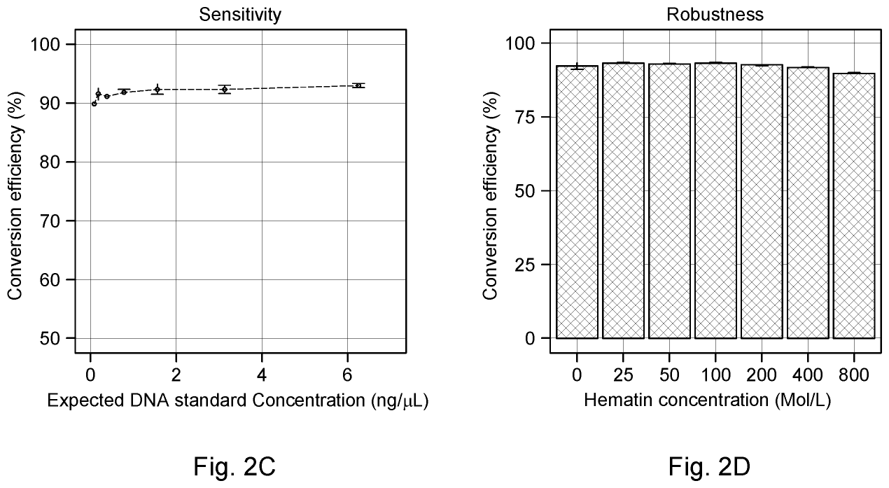

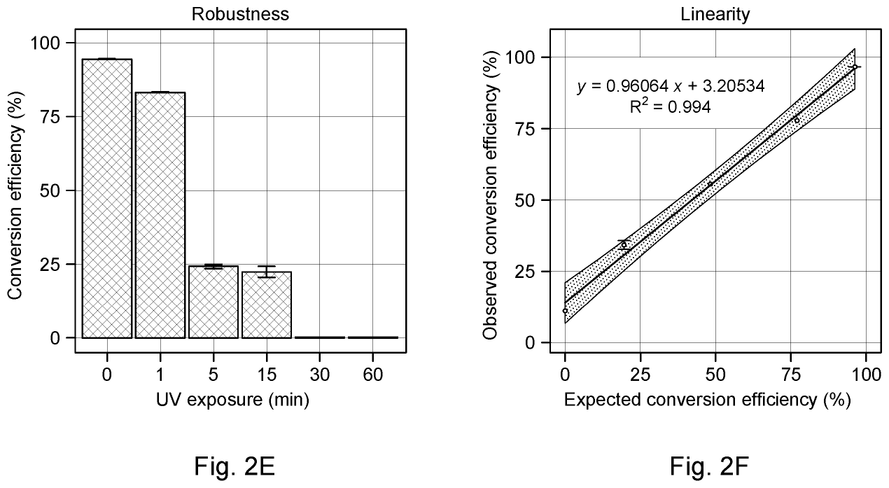

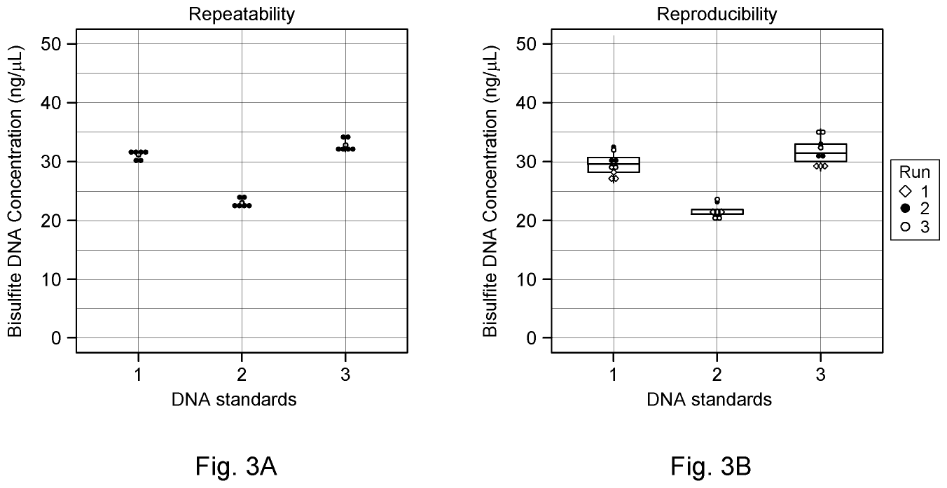

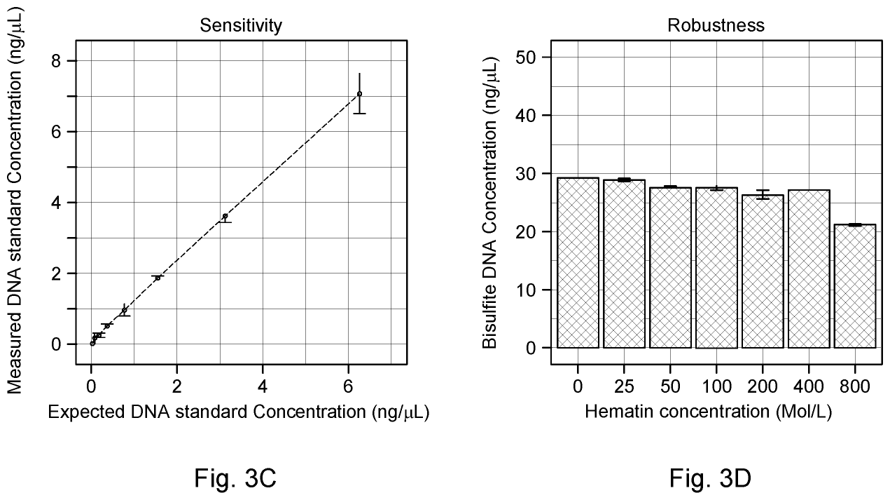

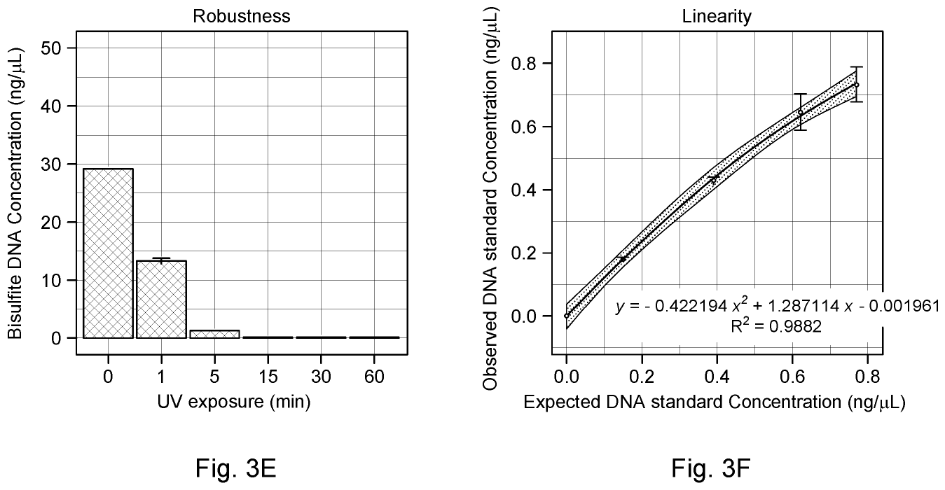

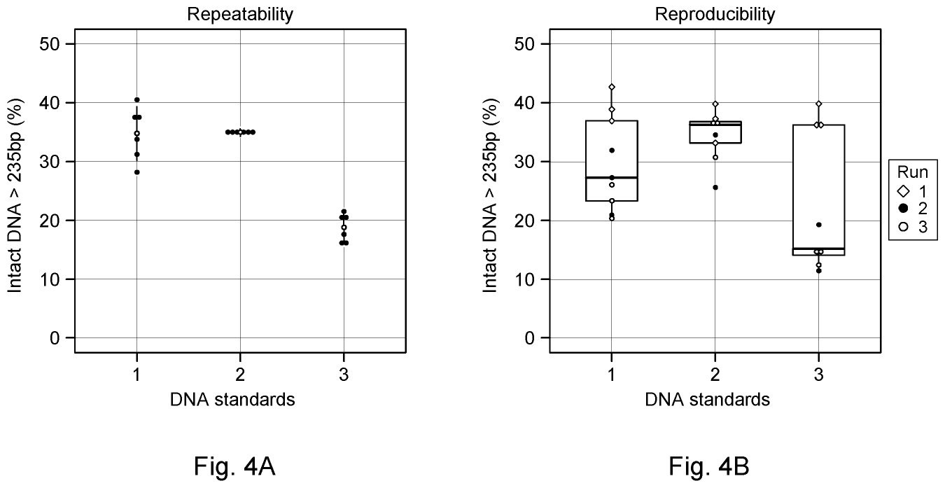

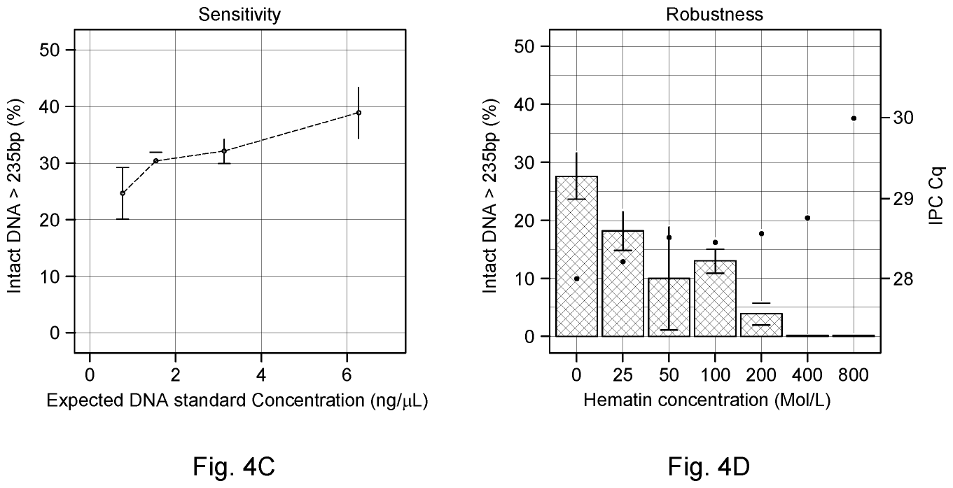

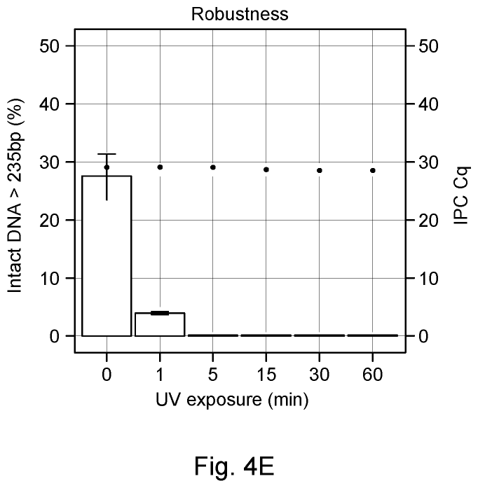

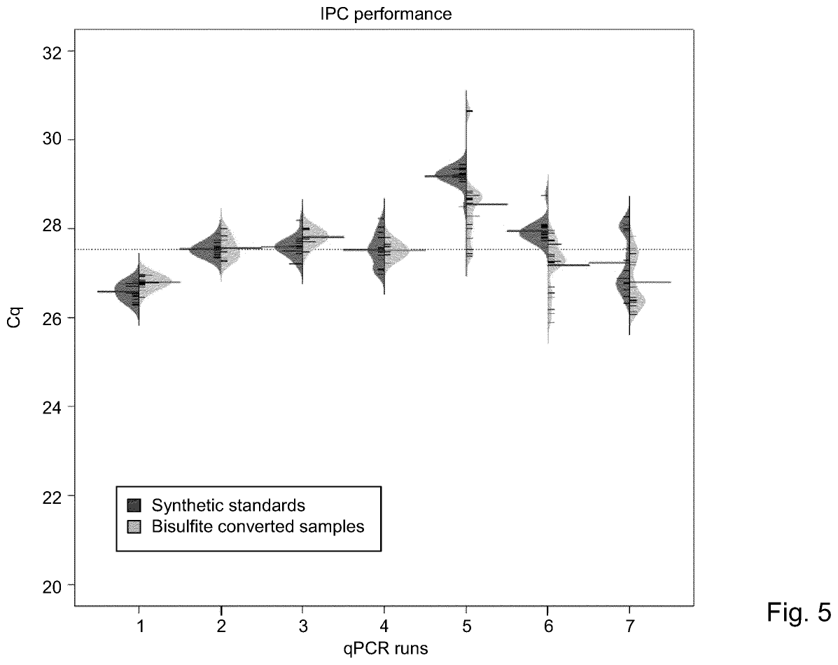

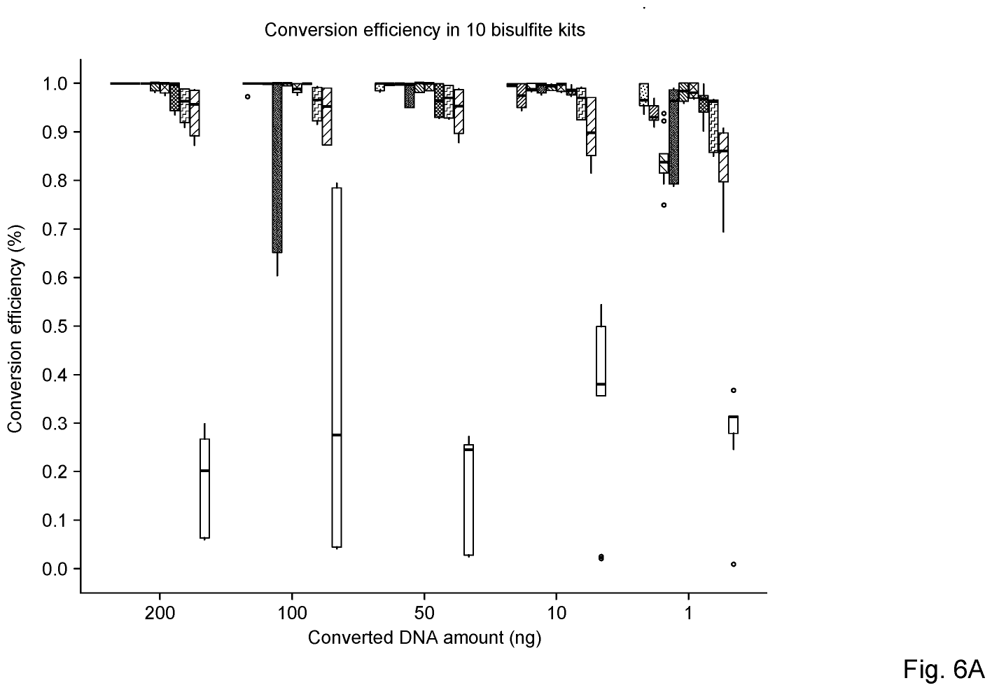

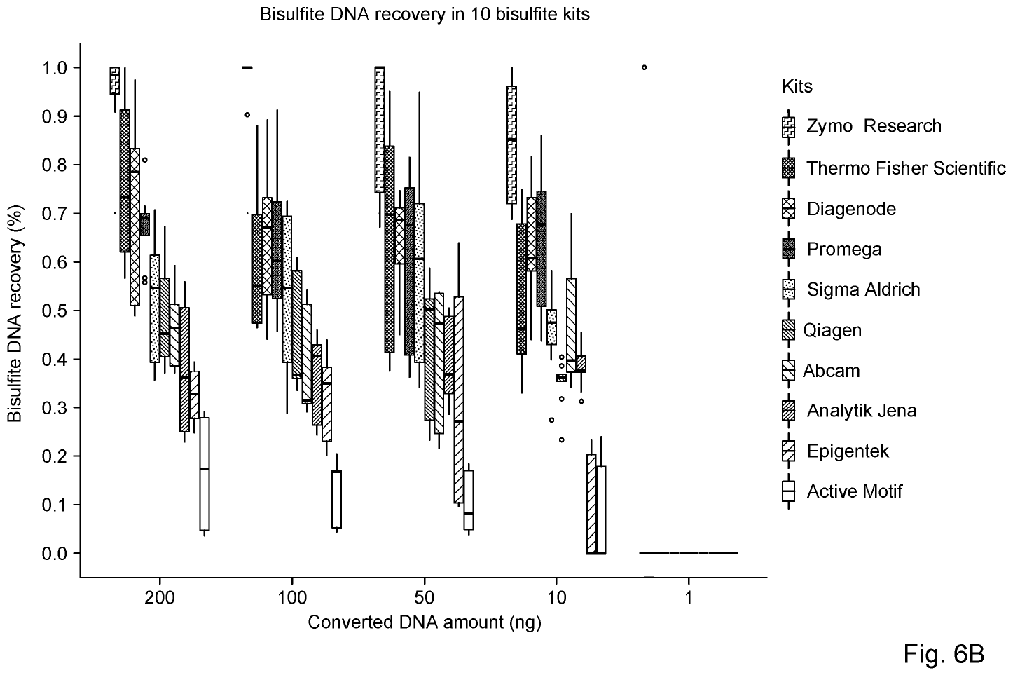

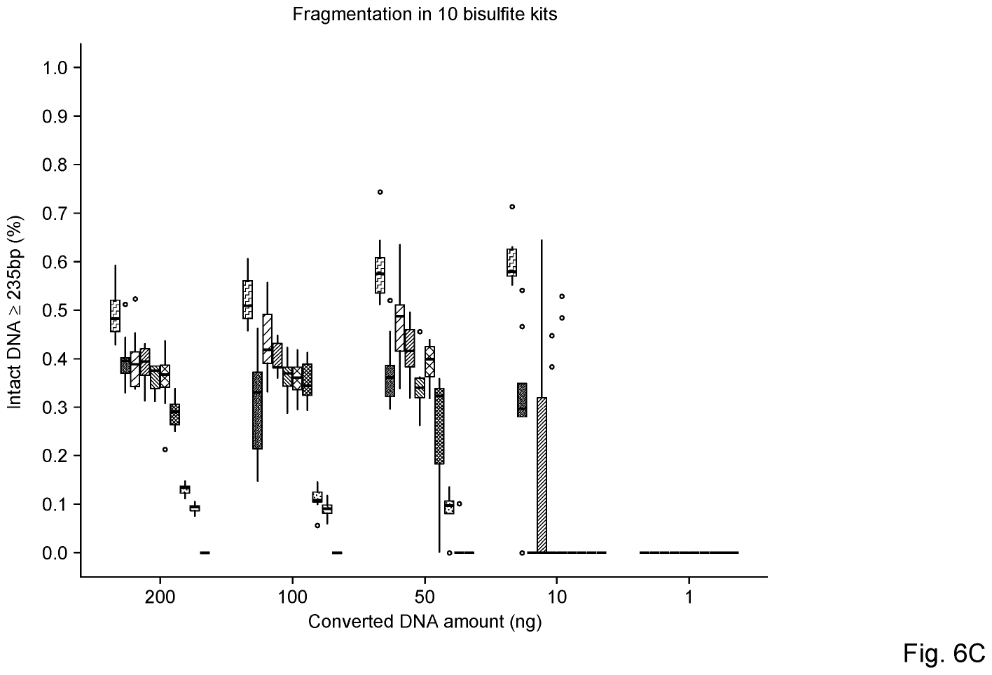

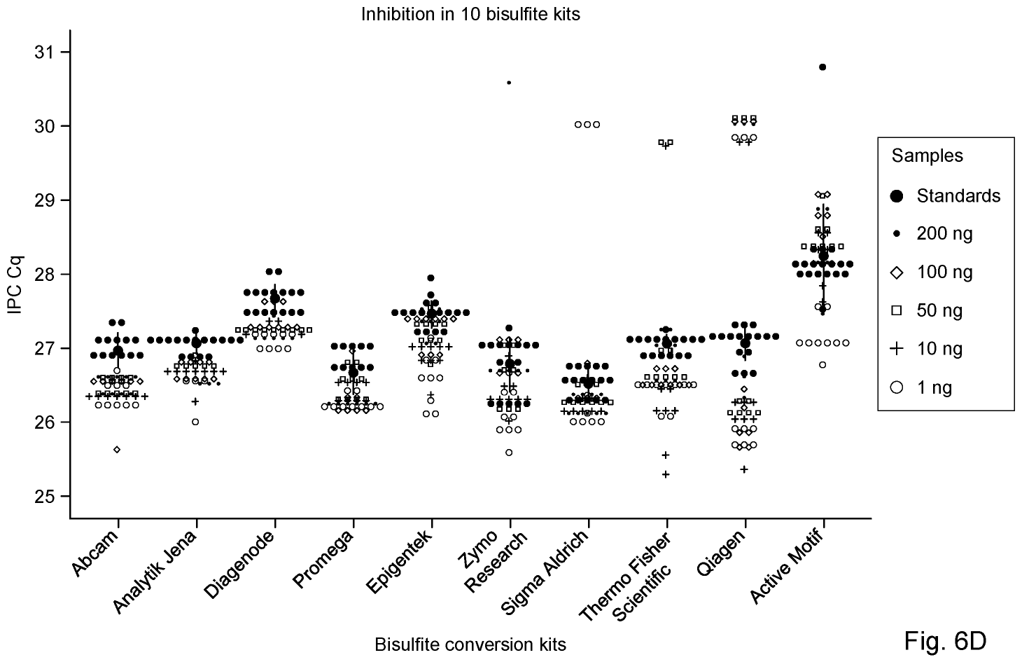

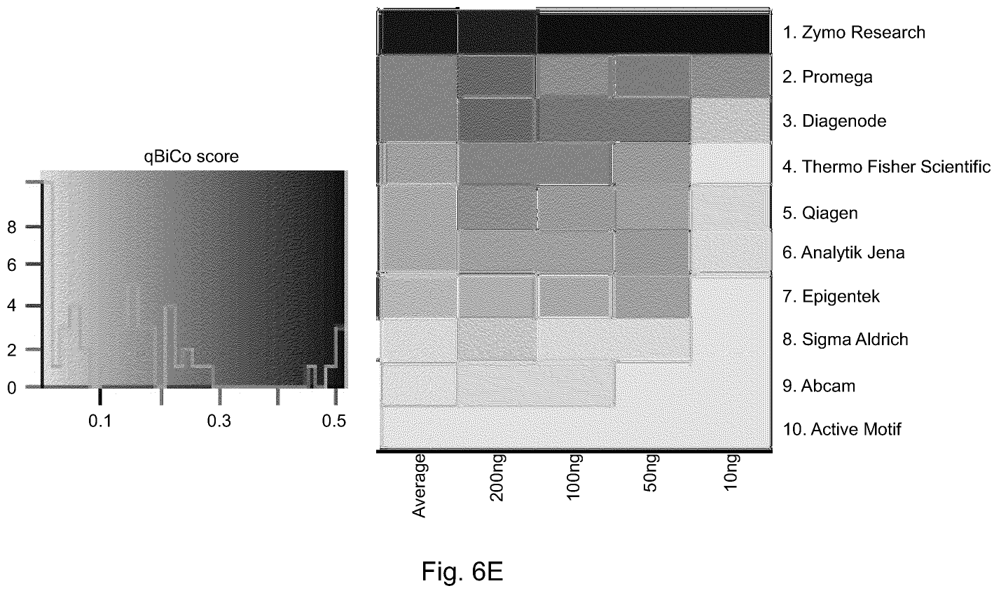

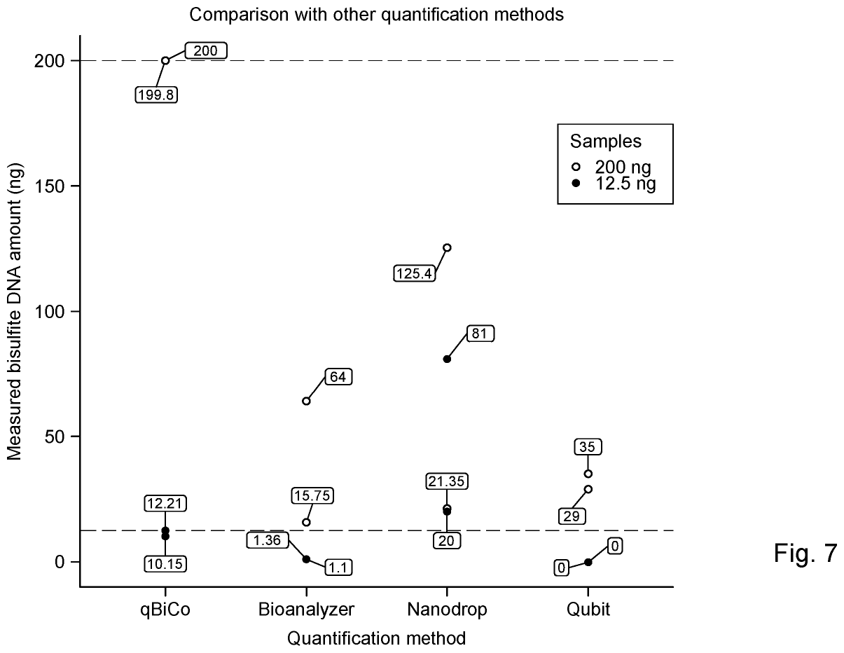

(A) Amplification curves of all five different qPCR assays included in qBiCo that indicates successful amplification of all DNA standards, (B) Amplification curves of the 5plex qBiCo assay of an example DNA standard (DNA standard 3 out of 8, based on gBlocks that correspond to 12.5 ng of bisulfite DNA) that indicates harmonized amplification of all assays in a single reaction, (C) Standard curves of the four assays (FAM, HEX, TEX615, Cy5) made by a dilution series (eight standards, 50-0.39 ng/μl) of the gBlock mix that is used for calculating the concentration of all four fragments in a sample (Note: No standard curve is required for IPC since the same amount, 10 ng is added to each reaction), (D) Internal Positive Control (IPC) performance taking into account all replicates of all eight synthetic DNA standards made by the dilution series of the gBlock mix at six different qPCR experiments during method development that indicates the average expected Cq of the assay (Note: if a sample's average IPC Cq is higher than the Average IPC of the eight synthetic standards plus standard deviation then we consider that the sample undergoes some degree of PCR inhibition (qualitative index, Yes/No), (E) Performance of 16 negative controls including no DNA template (only IPC) that indicates the Cq threshold to be used for qBiCo (32 cycles) to avoid potential contamination, (F) Heatmap of the limit of detection (50-0.39 ng/μl) of the four assays (gBlock standards of short hTERT, long hTERT, Line-1 converted, and Line-1 genomic, labelled, FAM, HEX, TEX615, Cy5) based on what was detected in the synthetic DNA standards with known expected concentrations that indicate high sensitivity for all fragments, (G) Assay efficiency using the same DNA standards within a timeframe of five days that indicates that DNA standards are stable can be used up to two days.

FIG. 2. qBiCo Method Validation with Regards to Estimating Bisulfite Conversion Efficiency of a Sample Using the Kit from ZymoResearch

(A) Conversion efficiency as determined by qBiCo for three different bisulfite-converted DNA samples (ZymoResearch, EpiGenDX and ThermoFisher, 200 ng used for bisulfite conversion which is the optimal amount) in six replicates within the same qPCR run that indicates high repeatability, (B) Conversion efficiency as determined by qBiCo using the same three different bisuflite-converted DNA samples in three replicates within three different qPCR runs that indicates high reproducibility, (C) Conversion efficiency as determined by qBiCo for a bisulfite-converted DNA sample (EpigenDx) that has been serially diluted in concentrations ranging from 6.25-0,048 ng/ul, indicating high sensitivity, (D) Conversion efficiency as determined by qBiCo for one bisulfite-converted DNA sample (EpigenDx) that have been exposed to seven different concentrations of hematin (0-800 mol/L) which is a known PCR inhibitor, that indicates ability to accurately calculate conversion efficiency regardless potential PCR inhibition, (E) Conversion efficiency as determined by qBiCo for a bisulfite-converted DNA sample (EpigenDx) after initial exposure prior to conversion (genomic DNA) to UV for six different times ranging from 0 to 60 minutes that indicates conversion efficiency calculations are influenced by direct UV exposure of >1 min due to DNA degradation, (F) Conversion efficiency as determined by qBiCo for five bisulfite-converted samples containing different ratios of converted/non-converted DNA ranging from 0-100% converted DNA that indicates linear quantification of conversion efficiency The artificial mixtures were created by bisulfite-converting 200 ng of the EpigenDx high methylated sample, quantifying the non-converted and converted DNA concentration with qBiCo using the Line-1 genomic and Line-1 converted assays respectively, diluting in equal concentration (2 ng/μl), mixing in the respective ratios, and using 1 μl corresponding to 2 ng in the qBiCo reaction). In FIGS. 2, 3, and 4, the term DNA standards under Panels A and B refers to different DNA samples.

FIG. 3. qBiCo Method Validation with Regards to Estimating Bisulfite DNA Recovery of a Sample Using the Kit from ZymoResearch

(A) Bisulfite DNA recovery as determined by qBiCo for three different bisulfite-converted DNA samples (ZymoResearch, EpiGenDX and ThermoFisher, 200 ng used for bisulfite conversion which is the optimal amount) in six replicates within the same qPCR run that indicates high repeatability, (B) Bisulfite DNA recovery as determined by qBiCo using the same three different bisuflite-converted DNA samples in three replicates within three different qPCR runs that indicates high reproducibility, (C) Bisulfite DNA recovery as determined by qBiCo for a bisulfite-converted DNA sample (EpigenDx) that has been serially diluted in concentrations ranging from 6.25-0,048 ng/ul, indicating high sensitivity, (D) Bisulfite DNA recovery as determined by qBiCo for one bisulfite-converted DNA sample (EpigenDx) that have been exposed to seven different concentrations of hematin (0-800 mol/L) which is a known PCR inhibitor, that indicates ability to accurately calculate conversion efficiency regardless potential PCR inhibition, (E) Bisulfite DNA recovery as determined by qBiCo for a bisuflite-converted DNA sample (EpigenDx) after initial exposure prior to conversion (genomic DNA) to UV for six different times ranging from 0 to 60 minutes that indicates severe Dna fragmentation affecting the amplification of the short fragment following direct UV exposure of >1 min, (F) Bisulfite DNA recovery as determined by qBiCo for five bisulfite-converted samples containing different ratios of converted/non-converted DNA ranging from 0-100% converted DNA that indicates second-degree multinomial quantification of bisulfite-converted DNA in line with previous literature reporting amplification bias between the non-converted vs converted version of a DNA sequence. The artificial mixtures were created by bisulfite-converting 200 ng of the EpigenDx high methylated sample, quantifying the non-converted and converted DNA concentration with qBiCo using the Line-1 genomic and Line-1 converted assays respectively, diluting in equal concentration (2 ng/μl), mixing in the respective ratios, and using 1 μl corresponding to 2 ng in the qBiCo reaction.

FIG. 4. qBiCo Method Validation with Regards to Estimating Intact Bisulfite DNA Amount (≥235 bp) of a Sample Using the Kit from ZymoResearch

(A) Intact bisulfite DNA recovery as determined by qBiCo for three different bisulfite-converted DNA samples (ZymoResearch, EpiGenDX and ThermoFisher, 200 ng used for bisulfite conversion which is the optimal amount) in six replicates within the same qPCR run that indicates moderate repeatability, (B) Intact bisulfite DNA recovery as determined by qBiCo using the same three different bisuflite-converted DNA samples in three replicates within three different qPCR runs that indicates moderate reproducibility, (C) Intact bisulfite DNA recovery as determined by qBiCo for a bisulfite-converted DNA sample (EpigenDx) that has been serially diluted in concentrations ranging from 6.25-0,048 ng/ul, indicating moderate sensitivity, (D) Intact bisulfite DNA recovery as determined by qBiCo for one bisulfite-converted DNA sample (EpigenDx) that have been exposed to seven different concentrations of hematin (0-800 mol/L) which is a known PCR inhibitor, that indicates potential PCR inhibition that influences the amplification of the long fragment, (E) Intact bisulfite DNA recovery as determined by qBiCo for a bisuflite-converted DNA sample (EpigenDx) after initial exposure prior to conversion (genomic DNA) to UV for six different times ranging from 0 to 60 minutes that indicates that more severe fragmentation of the long fragment compared to the short as one would expect following direct UV exposure of >1 min.

FIG. 5. qBiCo Validation with Regards to Estimating Potential PCR Inhibition of a Sample Using the Kit of ZymoResearch

Performance of the internal positive control (IPC) (10 ng used in each qPCR reaction) in seven different qPCR runs during qBiCo validation for both the eight synthetic DNA standards based on gBlocks (purple) and various bisulfite-converted DNA samples (green) (the number of samples in each run depends on which experiment was included in each run—i.e. one repeatability run, three reproducibility runs, one sensitivity run, etc without including the runs of artificially inhibited samples) that indicates high repeatability, reproducibility and ability to detect small degree of possible PCR inhibition in the bisulfite-converted DNA samples.

FIG. 6. qBiCo Application in Assessing Ten Different Commercially Available Bisulfite Conversion Kits Using Three Commercially Available DNA Standards

(A) Bisulfite conversion efficiency, (B) bisulfite DNA recovery, (C) bisulfite DNA fragmentation vs. starting DNA amount for bisulfite conversion (200 ng, 100 ng, 50 ng, 10 ng, 1 ng) as detected using qBiCo in ten bisulfite conversion kits that indicate high variation among kits for all three indexes (Note: 1 μl of the elution volume was used in each qPCR reaction), (D) Performance of the internal positive control (IPC) (10 ng added in each qPCR reaction) in ten bisulfite kits that indicates qBiCo can indicate potential inhibition in bisulfite-converted DNA samples, which is however scarce and kit-dependent, (E) Heatmap showing a kit ranking based on a qBiCo score, resulting from multiplying all three indexes—% bisulfite DNA recovery, % bisulfite conversion and % bisulfite intact DNA—that ranges between 0 and 1.

FIG. 7. qBiCo comparison with three other common DNA quantification methods with regards to bisulfite DNA recovery estimation for two bisulfite-converted DNA samples (200 ng and 12.5 ng, EpigenDx) that indicates qBiCo's superior performance.

The three other DNA quantification methods included 1) Bioanalyzer-Agilent RNA 6000 Pico kit, 2) Nanodrop— RNA mode, 3) Qubit—Qubit ssDNA DNA assay kit and following their standard manufacturer protocols. For all quantifications, 1 μl of the eluted bisulfite-converted DNA amount was used.

### DETAILED DESCRIPTION OF THE INVENTION

The term “nucleic acid sequence”, as used herein, refers to a DNA or RNA molecule in single- or double-stranded form.

An “isolated nucleic acid sequence” refers to a nucleic acid sequence which is no longer in the natural environment from which it was isolated. The term inter alia refers to a nucleic acid molecule that has been separated from at least about 50%, 75%, 90%, or more of proteins, lipids, carbohydrates, or other materials with which it is naturally associated, e.g. in a microbial host cell.

The term “isolating”, as used herein in the context of isolating nucleic acid sequences from a biological sample, refers to an in vitro process wherein nucleic acids, preferably genomic DNA, are extracted from a sample of interest. The process generally involves lysis of a (cells in) biological sample using a guanidine-detergent lysing solution that permits selective precipitation of DNA from a (cell) lysate, and precipitation of the genomic DNA from the lysate with ethanol. Following an ethanol wash, precipitated DNA may be solubilized in either water or 8 mM NaOH and used as template in a PCR reaction. Genomic DNA samples may be obtained by using generally known techniques for DNA isolation. The total genomic DNA may be purified by using, for instance, a combination of physical and chemical methods. Very suitably commercially available systems for DNA isolation and purification may be used.

The term “sample”, as used herein, includes reference to a sample of urine, saliva, sputum, pus, wound fluid, feces, skin, liquor, blood, a lavage, a biopsy, preferably of the human body, or an environmental sample, or a sample of a plant, an animal, or a food item.

The term “genomic DNA sample”, as used herein, includes reference to a sample that contains genomic DNA. Preferably, the same is purified to the extent that it is suitable for use in qPCR. In other words, preferably, genomic DNA has been at least partially been isolated from a more complex sample.

The term “methylated” or “methylation”, as used herein in reference to the methylation status of a cytosine, e.g., in a CpG locus or island, generally refers to the presence or absence of a methyl group at position 5 of the cytosine residue (i.e., whether a particular cytosine is 5-methylcytosine). Methylation can be determined directly, e.g., as evidenced by routine methods for analysis of methylation status of cytosines, e.g., by determining the sensitivity (or lack thereof) of a particular C-residue to conversion to uracil by treatment with bisulfite. For example, a cytosine residue in a sample that is not converted to uracil when the sample is treated with bisulfite in a manner that would be expected to convert that residue if non-methylated (e.g., under conditions in which a majority or all of the non-methylated cytosines in the sample are converted to uracils) may generally be deemed “methylated”.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like).

The term “target” when used in reference to a nucleic acid detection or analysis method, refers to a nucleic acid having a particular sequence of nucleotides to be detected or analyzed, e.g., in a sample suspected of containing the target nucleic acid.

The term “bisulfite conversion”, as used herein, refers to the process of treating DNA with bisulfite under conditions such that unmethylated cytosine residues in the DNA are converted to uracil but methylated cytosine residues (5-methylcytosine) are not converted to uracil. The bisulfite treatment is commonly applied and can for instance be conducted in the following way: Genomic DNA is isolated, denaturated by NaOH, converted several hours by a concentrated (sodium) bisulfite solution and finally desulfonated and desalted (e.g.: Frommer et al.: A genomic sequencing protocol that yields a positive display of 5-methylcytosine residues in individual DNA strands. Proc Natl Acad Sci USA. 1992 Mar. 1; 89(5):1827-31). Multiple commercial kits are available that provide for bisulfite conversion of DNA, including the EZ DNA Methylation Kit™ (ZymoResearch, USA).

The term “bisulfite conversion efficiency”, as used herein, refers to the efficiency with which the bisulfite conversion occurs. The efficiency can be expressed as the proportion of converted copies versus the total copies including both converted and unconverted copies of a (highly) multi-copy DNA sequence that is a repetitive DNA element.

The term “amplified” as used herein includes reference to subjecting a target nucleic acid in a sample to a process that linearly or exponentially generates amplicon nucleic acids having the same or substantially the same nucleotide sequence as the target nucleic acid, or segment thereof. The term “amplified” as used herein can refer to subjecting a target nucleic acid (e.g., in a sample comprising other nucleic acids) to a process that selectively and linearly or exponentially generates amplicon nucleic acids having the same or substantially the same nucleotide sequence as the target nucleic acid, or segment thereof. The term “amplified” includes reference to a method that comprises a polymerase chain reaction (PCR), more preferably qPCR.

The term “polymerase chain reaction (PCR)”, as used herein, refers to the well-known in vitro technique to make numerous copies of a specific segment of target DNA from a template DNA—i.e., the DNA that contains the target region to be copied. During the reaction a mixture containing the target DNA, primers, dNTPs, and a heat-stable DNA polymerase is heated to 90-95° C. to denature the strands of the target DNA. The solution is cooled to a temperature that allows the primers (single-stranded DNA molecules of about 18 to 30 nucleotides long) to anneal to their complementary sequence on the target DNA and provide the 3′-OH required for DNA synthesis. Subsequently, the DNA polymerase synthesizes a new DNA strand complementary to the target by extending the primer, usually at a temperature of about 72° C. The thermal cycling scheme of denaturing/primer annealing/primer extension is repeated numerous times with the DNA synthesized during the previous cycles serving as a template for each subsequent cycle. The result is a doubling of the target DNA present with each cycle, and exponential accumulation of target DNA sequences over the course of 20-40 cycles. A heating block with an automatic thermal cycler is used for precise temperature control. A preferred method for use in the present invention is qPCR amplification (also known as real-time PCR), wherein typically the amplification of a targeted DNA molecule is monitored during the PCR (i.e., in real time), using non-specific fluorescent dyes that intercalate with any double-stranded DNA or sequence-specific DNA probes consisting of oligonucleotides that are labelled with a fluorescent reporter for the detection of PCR products in real-time.

The term “qPCR”, as used herein, generally refers to the PCR technique known as real-time quantitative polymerase chain reaction, quantitative polymerase chain reaction or kinetic polymerase chain reaction. This technique simultaneously amplifies and quantifies target nucleic acids using PCR wherein the quantification is by virtue of an intercalating fluorescent dye or sequence-specific probes which contain fluorescent reporter molecules that are only detectable once hybridized to a target nucleic acid.

The term “PCR mixture”, as used herein, refers to the small volume of biochemical reactants in aqueous liquid for performing the PCR reaction comprising the (genomic) template DNA comprising the target DNA sequence(s), a set of at least two oligonucleotide primers that hybridize to opposite strands of the target DNA sequence(s) and flank the region to be amplified, a thermo-stable DNA polymerase, the four deoxyribonucleoside triphosphates (dNTPs), Mg2+ ions, and, preferably, a target-sequence-specific oligonucleotides (DNA) probe labelled with a fluorescent reporter.

The term “template”, as used herein, refers to the nucleic acid from which the target sequence is amplified in a nucleic acid amplification reaction. The term “amplifiable template”, as used herein, refers to a template that, when amplified, results in a single amplicon. Amplifiable templates comprise primer binding sites for hybridization of amplification primers.

The term “amplification primers”, as used herein, refers to the oligonucleotide primers that hybridize to opposite strands of the target DNA sequence(s) and flank the region to be amplified. During amplification, a primer anneals to its specific primer-binding site on the target DNA, and primer extension occurs while polymerase moves along the template strand in a 3′-5′ direction, resulting in formation of the daughter strand in a 5′-3′ direction.

The term “hybridize” as used herein generally refers to the base-p airing between different nucleic acid molecules consistent with their nucleotide sequences. The terms “hybridize” and “anneal” can be used interchangeably.

The term “complementary” as used herein generally refers to the ability to form favorable thermodynamic stability and specific pairing between the bases of two nucleotides at an appropriate temperature and ionic buffer conditions. This pairing is dependent on the hydrogen bonding properties of each nucleotide. The most fundamental examples of this are the hydrogen bond pairs between thymine/adenine and cytosine/guanine bases.

The terms “amplification product”, and “amplicon”, as used interchangeably herein, refer to a (usually double stranded) nucleic acid fragment that is the product of a nucleic acid amplification or replication event, such as for instance formed in the polymerase chain reaction (PCR). The term “PCR amplicon”, as used herein, refers to the PCR product or amplified target DNA.

The term “qPCR Cq value”, or, briefly, “Cq value”, as used herein, refers to the cycle value at which the (baseline-corrected) amplification curve (of fluorescence readouts generated from the PCR product in the real-time PCR reaction) crosses an arbitrary threshold value, indicating that amplification of the target occurs (ie. through exponential increase in target copy number).

The term “repetitive DNA element”, as used herein, refers to a DNA sequence of which multiple copies are present throughout the genome. The term “repetitive DNA element” includes reference to transposons such as a long interspersed nuclear element (LINE); (non-autonomous) small interspersed nuclear element (SINE) such as an Alu DNA element; and long terminal repeat (LTR) retrotransposons. Preferably, the repetitive DNA element has an element length (that is repeated) of at least 100 bp, more preferably at least 400 bp, even more preferably at least 1 kb or more preferably at least 2 kb long or at least 4 kb, and most preferably 6-7 kb. Preferably, the repetitive DNA element is a (human) LINE, including LINE1, LINE2 and LINE3. Most preferably, the LINE is a LINE1.

Preferably, said repetitive DNA element has 10-1000 copies throughout the genome, preferably human genome, more preferably 25-800 or 50-800 copies, even more preferably 100-500 copies and most preferably 150-450 copies. The term “repetitive DNA element” can be used interchangeably with the terms “repeated sequence”, “repeated unit” or “repeats”.

The cycle in which fluorescence can be detected is termed quantitation cycle (Cq for short) and is the basic result of qPCR: lower Cq values mean higher initial copy numbers of the target. This is the basic principle of quantitative approach that real-time PCR provides.

### Bisulfite Conversion Efficiency

The methods of the invention employ qPCR as a means for assaying. qPCR is a well-known analytical method in the art. The inventor unexpectedly established that especially a 5plex qPCR assay comprising the primers and probes identified by SEQ ID NOs:1-12 (preferably in addition thereto the primers and probes of SEQ ID NOs:13-15 if the internal positive control (IPC) identified by SEQ ID NO:20 is added to the qPCR mixture (this may be after the bisulfite treatment step such that the IPC is an unconverted sequence)), can be advantageously employed in simultaneously determining a bisulfite conversion efficiency, a bisulfite converted DNA quantity, a level of DNA fragmentation following bisulfite conversion and a level of PCR inhibition. Such an assay combines the possibility to assess all relevant reaction variables in a PCR reaction for subsequently studying DNA methylation based on bisulfite-converted DNA.

It is generally known that bisulfite treatment generally does not result in 100% conversion efficiency.

Preferably, in the method to determine global bisulfite conversion efficiency of the invention, a bisulfite-treated target DNA sequence that is a DNA repetitive element is amplified with two different sets of amplification primer. Preferably, both sets of amplification primers amplify at least partly the same target DNA sequence which contains cytosine residues that are converted to uracil when bisulfite conversion has occurred. Amplifying multiple copies of such a multi-copy gene region thus allows for discriminating between copies that are bisulfite-converted and copies that are not bisulfite-converted. Using primers and/or probes that discriminate between converted and unconverted copies of such a multi-copy DNA sequence allows for estimation of the global bisulfite conversion efficiency.

In a method of the invention, genomic DNA is preferably first chemically modified by sodium bisulfite (at 1-10M, preferably about 3-6M more preferably about 4-5M of sodium bisulfite, optionally provided in the form of sodium metabisulfite). This allows for the generation of bisulfite-converted sequence differences. Fluorescence-based qPCR can then be performed with primers that overlap non-CpG cytosines that are expected to be converted (both Line-1 primer annealing sites contain cytosines that are converted, but one of the provided primers of a set binds to the T and one of the primers of another set binds to the unconverted C (i.e. residual genomic)). Sequence discrimination can thus occur either at the level of the PCR amplification process or at the level of the probe hybridization process, or both. Sequence discrimination at the PCR amplification level requires the primers and/or probe to overlap potential non-CpG cytosines that are expected to be converted.

Preferably, in a method of the invention relating to determining global bisulfite conversion efficiency in a bisulfite-converted DNA sample, the detection probe provides for sequence discrimination (i.e. discrimination between bisulfite-converted copies and unconverted copies or, in other words, the first probe is specific for a bisulfite-converted copy and not for a bisulfite-unconverted copy whereas the second probe is specific for a bisulfite-unconverted copy and not for a bisulfite-converted copy). Alternatively, in a method of the invention relating to bisulfite conversion efficiency, the amplification primers provide for sequence discrimination (i.e. discrimination between bisulfite converted copies and unconverted copies or, in other words, the first primer pair is specific for a bisulfite converted copy and not for a bisulfite unconverted copy whereas the second primer is specific for a bisulfite unconverted copy and not for a bisulfite converted copy). More preferably, in a method of the invention relating to bisulfite conversion efficiency, both the amplification primers and the detection probe provide for sequence discrimination (i.e. discrimination between bisulfite converted copies and unconverted copies or, in other words, both the first primer pair and the first probe are specific for a bisulfite converted copy and not for a bisulfite unconverted copy whereas the second primer pair+second probe are specific for a bisulfite unconverted copy and not for a bisulfite converted copy).

In qPCR methods and kits of the invention, conventional heat-stable (Taq) polymerases may be used. Preferably, in such a qPCR methods and kits, a hot-start DNA polymerase is employed, for instance a hot-start Taq DNA Polymerase which is a mixture of Taq DNA Polymerase and an aptamer-based inhibitor. The aptamer-based inhibitor binds reversibly to the enzyme, inhibiting polymerase activity at temperatures below 45° C., but releases the enzyme during normal cycling conditions, allowing reactions to be set up at room temperature. Other suitable examples of hot start DNA polymerases that can be employed are e.g. AmpliTaq Gold® DNA Polymerase and Phusion® High-Fidelity DNA Polymerase.

Preferably, a method of the invention employs a qPCR kit of the invention as defined below. Preferably, primers and probe of SEQ ID NOs:7-12 are employed in a method of the invention (2 plex qPCR) for determining conversion efficiency as defined herein, more preferably primers and probes of SEQ ID NO:1-3 are employed in combination with primers and probes of SEQ ID NO: 7-12 in a method of the invention (3plex qPCR), even more preferably primers and probes of SEQ ID NOs:1-12 are employed in combination in a method of the invention (4plex qPCR), and most preferably primers and probes of SEQ ID NO:1-15 are employed in a method of the invention (5plex qPCR). In general, in a method of the invention, the qPCR DNA sample to be analysed, and that is to be treated or that has been treated with bisulfite, is spiked with a known amount of an internal positive control (IPC) comprising the DNA of SEQ ID NO:20.

In an alternative embodiment of a method or kit of the invention, the probe of SEQ ID NO:12 (probe for Line-1 genomic) can be replaced by TGGGAGTGACCCAATTTTCCAGGTG. In an alternative embodiment of a method or kit of the invention, the probe of SEQ ID NO:9 (probe for Line-1 converted) can be replaced by TGGGAGTGATTTAATTTTTTAGGTGY.

Unexpectedly, the inventor was able to design and successfully validate a 5plex a qPCR assay having all quantitative and qualitative effects mentioned herein, while amplifying a multi-copy target DNA sequence and creating a large amount of amplification product that did not hinder successful simultaneous detection and quantification of the other (single-copy number target sequences. The difference in copy number of DNA templates competing for the same reactants in the qPCR reaction may be as high as 200-fold.

Bisulfite conversion efficiency (ratio or %) can be calculated by any suitable formula available, based either on amount (in ng) or concentration (in ng/μl). These formulas are generally available to the skilled person. One suitable example is the formula: ([amount of first amplicon (ng)]/[amount of first amplicon (ng)+amount of second amplicon (ng)]).

The skilled person is well-aware how to employ standard curves in qPCR in order to calculate an amount of target DNA in a subsequent sample.

### DNA Recovery

The inventor established that by capitalizing on converted-specific sequence differences after bisulfite treatment, it is possible to determine bisulfite-converted DNA quantity. This is done by using a third set of primers and/or a third probe that is/are specific for bisulfite-converted DNA of a single-copy gene sequence.

Preferably, in such a method, the third detection probe provides for sequence/amplicon discrimination (i.e. the third detection probe is specific for bisulfite-converted DNA and allows for detecting and quantifying only bisulfite-converted DNA and not bisulfite-unconverted DNA). Alternatively, in such a method, the third set of amplification primers provide for sequence discrimination (i.e. the third primer set is specific for converted DNA and allows for detecting and allowing for the quantification of only bisulfite-converted DNA and not bisulfite unconverted DNA). More preferably, in such a method, both the third set of amplification primers and the third detection probe provide for sequence discrimination (i.e. both the third primer set and third probe are specific for bisulfite converted DNA and allows for the detection and quantification of only converted DNA). Essentially this is true for all sets of amplification primers/probes that are to amplify, detect and quantify converted DNA.

Preferably, the amplicon amplified by the third primer set is relatively short, preferably 70-150 bps, more preferably 80-130 bps, even more preferably 90-100 bps and most preferably about 85 bps. To achieve this, one may one may suitably design the third primer set for amplifying only a relatively short fragment (e.g. between 70 and 150 bp, suitably about 75-100 bp, preferably about 85 bps) from a genomic single copy DNA locus. By thus amplifying a short fragment of DNA of a single-copy DNA locus, one may accurately quantify the total amount of bisulfite-converted DNA in a sample. Preferably, the amplification target for quantifying DNA recovery following bisulfite-conversion is a putative single-copy gene or single-copy target locus such as human telomerase reverse transcriptase gene (hTERT), SRY (Y chromosome), ribonuclease P/MRP 30 kDa subunit (RPP30), vimentin (Vim), albumin (ALB), gamma globin (HBG), cyclin dependent kinase 6 (CDK6) or RNase P (RPPH1). Preferably only a portion of the single-copy DNA locus is amplified. The advantage of using putative single-copy genes for this purpose is their uniqueness and high sequence conservation. hTERT and RNase P are regularly used as endogenous reference genes in commercially available human qPCR quantification kits.

The term “single-copy gene” refers to genes and sequences thereof that (putatively) have one single physical location in the genome and may have orthologs in different species. One example of such a single copy gene is (human) telomerase reverse transcriptase gene (hTERT).

Preferably, in aspects of this invention, the putative single-copy gene is an autosomal gene, most preferably hTERT is used as the putative single-copy gene for determining DNA recovery. The portion of the human telomerase reverse transcriptase gene (hTERT), located on 5p 15.33, is preferably amplified using the primers of SEQ ID NO:1 and 2 as amplification primers, with SEQ ID NO:3 as the probe sequence. For accurate quantitation of the amount of bisulfite-converted DNA in the sample, the skilled person is aware that diploid cells contain two copies (alleles) of the single-copy genes, one per chromosome.

The bisulfite DNA concentration in the sample subjected to qPCR equals the concentration of the short fragment of bisulfite-converted DNA of a single-copy DNA locus in the sample subjected to qPCR (Conc (Short)).

### Fragmentation Level

For quantifying degradation due to bisulfite treatment, which is a common phenomenon due to the high temperature and harsh chemical conditions, an additional primer set is used for amplifying a longer target sequence of the same single-copy DNA locus from which a short fragment was amplified for the purpose of quantifying DNA recovery described above. Hence, a short fragment (e.g. 85 bp as exemplified for the embodiment for hTERT short herein) of the single-copy DNA locus may be used herein to quantify the total amount of bisulfite-converted DNA amount (using the third set of primers as defined herein), whereas a longer fragment ((e.g. 235 bp as exemplified for the embodiment for hTERT long herein) of the single-copy DNA locus may be used herein to quantify the potential degradation of DNA during bisulfite conversion. Hence, the quality of the DNA may suitably be expressed as the amount or concentration of intact DNA (e.g. DNA ≥235 bp) by dividing the amount or concentration of long DNA by the amount or concentration of short DNA (e.g. Intact DNA (≥235 bp)=Conc (Long)/Conc (Short).

In aspects of the invention, the detection probes can be labeled with numerous qPCR labels or dyes, including FAM, HEX, TEX, Cy5 and Cy5.5 dyes. Use can also be made of hydrolysis (e.g., TaqMan®) probes, incorporating a 5′ reporter fluorophore and a 3′ quencher on a short oligonucleotide complementary to the target sequence.

Preferably, the “long” amplicon (generated using the fourth primer and probe set as described herein) has length of 180-400 bps, more preferably 200-350 bps, even more preferably 220-300 bps and most preferably about 235 bps.

### PCR Inhibition

Finally, a fifth set of amplification primers can be employed to amplify an artificially designed, internal control DNA fragment (for instance, a DNA comprising SEQ ID NO:20) that is added to the qPCR mixture. This allows for determining the level of potential PCR inhibition. The skilled person is well-aware how to employ internal standards in a qPCR reaction. For the purpose of the present invention an artificial sequence was generated to function as an internal positive control (IPC) to assess the presence of sample inhibition. It is preferred that the sequence of this synthetic DNA is not found in the human genome. It is also preferred that the sequence of this artificial DNA is a synthetic DNA, preferably the DNA resembles the single-copy target locus short fragment as defined herein (e.g. the hTERT short fragment), which is used to quantify DNA recovery. A dedicated primer set may then be used to amplify this IPC sequence in a multiplex PCR according to the present invention, wherein these IPC primers bind to the IPC fragment, and the amplicon may be detected by use of yet another probe that is fluorescently labelled, for instance with a Cy5.5™ dye. In order to ensure that the IPC sequence is only amplified from the IPC template DNA and not from the (human) genomic DNA or converted DNA present in the PCR reaction, and to prevent binding of other primers in the multiplex PCR reaction of the invention to the IPC template DNA sequence, the IPC sequence preferably is not found in nature. This may be evaluated, for instance by performing a BLAST search, well known to the skilled person. Also for the probe for the IPC amplicon, as for each of the other probes in the present invention, the probe is preferably designed to bind in a region in the synthetic sequence.

Primers and probes may be designed using dedicated software packages, such as Bisearch software. It is preferred that the primers are designed in a bisulfite-converted region without CpGs, or with as few CpGs as possible, in which case a ‘Y’ is added in the sequence of the primers to achieve successful and unbiased binding regardless of the methylation status. For CpG-rich fragments it may not always be possible to find primer sets which generate a single PCR product without any CpG. In the context of the hTERT long sequence for determining level of DNA fragmentation, the PCR product of the longer fragment contained 1 CpG site. In order to identify suitable target regions for the probes, the PCR products may be converted in silico with aid of Methylprimer software. It is preferred that the probe annealing sites contain as many converted cytosines as possible. Once suitable target sites for amplification have been identified, the predicted amplicons may be checked for the presence of SNPs in that region. For this, one may use the online-source Ensemble. Candidate primers and probes are preferable checked for formation of hairpins and primer dimers. For this, one may use can be made of software package Autodimer V1.

Typically, in aspects of this invention, the amount of DNA used as IPC, and preferably the amount of DNA used as template in the multiplex PCR of the present invention is about 10 ng/μl of DNA.

### Kits of the Invention

A qPCR kit for assessing bisulfite conversion efficiency following bisulfite conversion of unmethylated cytosine to uracil in a genomic DNA sample by using the methods of the present invention typically comprises:
a first set of amplification primers for amplifying bisulfite converted copies of a genomic repetitive DNA element in said genomic (preferably human) DNA sample by qPCR and for generating a first amplicon; and a first detection probe labeled with a first detectable label for detecting said first amplicon by qPCR;
providing a second set of amplification primers for amplifying unconverted copies of said genomic repetitive DNA element in said genomic DNA sample by qPCR and for generating a second amplicon; and a second detection probe labeled with a second detectable label for detecting said second amplicon by qPCR;
optionally the kit comprises qPCR reagents (also known as Mastermix components), including, but not limited to reagents selected from a DNA polymerase, dNTPs, MgCl, and PCR buffer.

The set of amplification primers in aspects of this invention may comprise a first primer from a first set of two primers for primer extension in a qPCR reaction from a fully complementary binding site in a converted DNA template that is a repetitive element in a genomic DNA as defined herein. This primer extension reaction will provide the complementary strand of the converted DNA template, which complementary strand serves as the template for the second primer from the first set of two primers.

The set of amplification primers in aspects of this invention may comprise a first primer from a second set of two primers for primer extension in a qPCR reaction from a fully complementary binding site in a unconverted (genomic) DNA template that is a repetitive element in a genomic DNA as defined herein. This primer extension reaction will provide the complementary strand of the unconverted (genomic) DNA template, which complementary strand serves as the template for the second primer from the second set of two primers.

In aspects of this invention, the first and second set of primers are employed in a multiplex qPCR for generating a first amplicon with said first primer set, and, if unconverted (genomic) copies are present, said second amplicon with said second primer set.

The kit of the present invention provides detection probes for detecting the amplicon generated from the first and second set of primers as defined herein, by qPCR. Said probes are fluorescently labeled.

A kit of the present invention may further comprise;
a third set of amplification primers for amplifying a part of a bisulfite converted genomic single copy DNA locus in said genomic (preferably human) DNA sample (preferably hTERT as described above, although alternative targets are envisioned herein), by qPCR and for generating a third amplicon; and a third detection probe labeled with a third detectable label for detecting said third amplicon by qPCR, as described hereinabove; and
a fourth set of amplification primers for amplifying by qPCR a part of said single copy DNA locus that is bisulfite-converted and for generating a fourth amplicon that is longer in length than said third amplicon; and a fourth detection probe labeled with a fourth detectable label for detecting said fourth amplicon by qPCR; The terms first, second, third, fourth and fifth, as used herein, indicate that the elements differ.

Optionally the kit may comprise DNA standards or references as described herein, including DNA sequences comprising any of the gBlocks as described herein, preferably SEG ID NO:20, which is the internal positive control.

Preferably, a kit of the invention comprises a qPCR kit. Preferably, said kit comprises primers and probe of SEQ ID NOs:7-12 (a 2 plex qPCR kit) for determining conversion efficiency as defined herein, more preferably primers and probes of SEQ ID NO:1-3 are comprised in said kit in combination with primers and probes of SEQ ID NO: 7-12 (a 3plex qPCR kit), even more preferably primers and probes of SEQ ID NOs:1-12 are comprised in combination in a kit of the invention (a 4plex qPCR kit), and most preferably primers and probes of SEQ ID NO:1-15 are comprised in a kit of the invention (a 5plex qPCR kit). Most preferably, a kit of the invention further comprises an internal positive control (IPC) comprising the DNA of SEQ ID NO:20.

The invention also provides a qPCR kit comprising—a first set of amplification primers for amplifying bisulfite converted copies of a genomic multi-copy target DNA sequence that is a repetitive DNA element and for generating a first amplicon, preferably wherein said first set of amplification primers comprises a forward primer comprising the nucleotide sequence of SEQ ID NO:7 and a reverse primer comprising the nucleotide sequence of SEQ ID NO:8. Said qPCR kit may further comprise a first detection probe labeled with a first detectable label for detecting said first amplicon, preferably wherein said first detection probe comprises the nucleotide sequence of SEQ ID NO:9, more preferably wherein said first detection probe comprises the nucleotide sequence of SEQ ID NO:9 and a 5′TEX 615 dye.

Preferably, a qPCR kit of the invention further comprises—a second set of amplification primers for amplifying unconverted copies of said repetitive DNA element and for generating a second amplicon; preferably wherein said second set of amplification primers comprises a forward primer comprising the nucleotide sequence of SEQ ID NO:10 and a reverse primer comprising the nucleotide sequence of SEQ ID NO:11. Said qPCR kit may further comprise a second detection probe labeled with a second detectable label for detecting said second amplicon, preferably wherein the second detection probe comprises the nucleotide sequence of SEQ ID NO:12, more preferably wherein said second detection probe comprises the nucleotide sequence of SEQ ID NO:12 and a 5′Cy5™ dye.

It is preferred that said first set of amplification primers and said second set of amplification primers are included in said qPCR kit. In addition, it is preferred that both said first and said second detection probes are included in said kit.

Preferably, a qPCR kit of the invention further comprises a third set of amplification primers for amplifying a part of a genomic single copy gene sequence that is bisulfite converted and for generating a third amplicon, preferably wherein said third set of amplification primers comprises a forward primer comprising the nucleotide sequence of SEQ ID NO:1 and a reverse primer comprising the nucleotide sequence of SEQ ID NO:2. Said qPCR kit may further comprise a third detection probe labeled with a third detectable label for detecting said third amplicon, preferably wherein said third detection probe comprises the nucleotide sequence of SEQ ID NO:3, more preferably wherein said third detection probe comprises the nucleotide sequence of SEQ ID NO:3 and a ZEN™ quencher and a 5′FAM™ dye.

It is preferred that said first set of amplification primers, said second set of amplification primers and said third set of amplification primers are included in said qPCR kit. In addition, it is preferred that said first, said second and said third detection probes are included in said kit.

Preferably, a qPCR kit of the invention further comprises a fourth set of amplification primers for amplifying a part of said genomic single copy gene sequence that is bisulfite converted and for generating a fourth amplicon that is longer in length than said third amplicon; preferably wherein said fourth set of amplification primers comprises a forward primer comprising the nucleotide sequence of SEQ ID NO:4 and a reverse primer comprising the nucleotide sequence of SEQ ID NO:5. Said qPCR kit may further comprise a fourth detection probe labeled with a fourth detectable label for detecting said fourth amplicon, preferably wherein said fourth detection probe comprises the nucleotide sequence of SEQ ID NO:6, more preferably wherein said wherein said fourth detection probe comprises the nucleotide sequence of SEQ ID NO:6 and a ZEN™ Quencher and a 5′HEX™ dye.

It is preferred that said first set of amplification primers, said second set of amplification primers, said third set of amplification primers and said fourth set of amplification primers are included in said qPCR kit. In addition, it is preferred that said first, said second, said third and said fourth detection probes are included in said kit.

Preferably, said qPCR kit further comprises: —a first synthetic DNA standard consisting of the nucleotide sequence of said first amplicon, preferably wherein said first synthetic DNA standard is the sequence of SEQ ID NO:18; and—a second synthetic DNA standard consisting of the nucleotide sequence of said second amplicon, preferably wherein said second synthetic DNA standard is the sequence of SEQ ID NO:19; and/or; —a third synthetic DNA standard consisting of the nucleotide sequence of said third amplicon, preferably wherein said third synthetic DNA standard is the sequence of SEQ ID NO:16; and/or—a fourth synthetic DNA standard consisting of the nucleotide sequence of said fourth amplicon, preferably wherein said fourth synthetic DNA standard is the sequence of SEQ ID NO:17.

Preferably, a qPCR kit of the invention further comprises a fifth set of amplification primers for amplifying an artificial DNA sequence (or an internal positive control DNA sequence) and for generating a fifth amplicon; preferably wherein said fifth set of amplification primers comprises a forward primer comprising the nucleotide sequence of SEQ ID NO:13 and a reverse primer comprising the nucleotide sequence of SEQ ID NO:14. Said qPCR kit may further comprise a fifth detection probe labeled with a fifth detectable label for detecting said fifth amplicon by qPCR, preferably wherein said fifth detection probe comprises the nucleotide sequence of SEQ ID NO:15, more preferably wherein said fifth detection probe comprises the nucleotide sequence of SEQ ID NO:15 and a 5′Cy5.5™ dye.

It is preferred that said first set of amplification primers, said second set of amplification primers, said third set of amplification primers, said fourth set of amplification primers and said fifth set of amplification primers are included in said qPCR kit. In addition, it is preferred that said first, said second, said third, said fourth and said fifth detection probes are included in said kit.

It is further preferred that a qPCR kit of the invention comprises a nucleotide sequence that comprises an internal positive control for amplification, preferably a nucleotide sequence comprising SEQ ID NO:20.

The skilled person understands that the fifth set of amplification primers and fifth probe are matched to the internal positive control nucleotide sequence that is SEQ ID NO:20. In principle, the skilled person can also use alternative internal positive control nucleotide sequence and amplification primers and probes that are matched to said (i.e. amplify) said internal positive control nucleotide sequence.

Preferably, in a method of the invention, a qPCR kit of the invention is employed. Embodiments of the qPCR kit described in relation to a method of the invention also applies in relation to a qPCR kit of the invention.

The invention also provides a nucleic acid, preferably a synthetic oligonucleotide, comprising or consisting a sequence of SEQ ID NOs:1-20.

qBiCo

The present invention in one highly preferred embodiments provides methods and kits for performing a 5plex PCR reaction using specific primers and labeled probes (SEQ ID Nos: 1-15 as described herein) for determining various qualitative and quantitative aspects of bisulfite-converted DNA samples.

The qBiCo methods and kits, both in these highly preferred embodiments, but also in more general described aspects, comprising primers and probes for detecting bisulfite conversion efficiencies based on measuring converted and unconverted amplicons of a repetitive DNA element, preferably LINE-1, can be applied in many scientific areas including epigenetics, that employ bisulfite conversion of DNA. This can involve not only fundamental research and large-data epigenomic studies, i.e. large cohort studies that investigate DNA methylation patterns that use either Illumina microarray platforms (450K/EPIC) or whole-bisulfite sequencing technology to analyse hundreds or thousands or samples, but also targeted, diagnostic research and applications i.e. groups developing or applying targeted diagnostics tools, such as for cancer detection and disease development evaluation. qBiCo can be introduced to standardize any bisulfite conversion-based method, independently of the downstream analysis platform, ranging from simple PCRs or complex next-generation sequencing.

The present invention now provides for means and methods for measuring or quantifying the global (genome-wide) bisulfite conversion efficiency. As indicated herein above, the means and methods preferably comprise the use of a single-copy gene as a target for quantification by qPCR of the amount of bisulfite converted DNA in a sample, and the use of a different-sized fragment of the same single-copy gene for quantification by qPCR of the extend of fragmentation of the bisulfite converted DNA in said sample. Preferably, as indicated herein above, the means and methods preferably comprise the use of an internal positive control (IPC) for determining potential PCR inhibition. The IPC is preferably a synthetic non-human DNA sequence.

As indicated herein above, the means and methods for measuring or quantifying global bisulfite conversion efficiency preferably comprise the use of artificial DNA fragments (“gBlocks”, as termed herein) as DNA template in reference samples, that are run in parallel to the test sample. These reference samples are preferably run with the same PCR mastermix, primers and probes, as those used in the test sample. The reference sample provides for the preparation of a reference or standard curve, comprising known amounts of the artificial DNA templates. In a first preferred embodiment, the reference sample comprises at least a first artificial DNA fragment comprising a sequence that resembles, or is, a (part of) fully bisulfide converted sequence of a human genomic nucleotide sequence of a repetitive DNA element, as well as a second artificial DNA fragment comprising a sequence that resembles, or is, (a part of) the human genomic nucleotide sequence of a repetitive DNA element.

The IPC is preferably a synthetic non-human DNA sequence that is added to each of the qPCR reactions (both references or standards, as well as test samples) in order to detect potential PCR inhibition.

The gBlocks artificial DNA fragments may comprise as synthetic DNA standards (references) the expected sequences of the PCR products as amplified using the various primers for amplifying the repetitive DNA element having a genomic (unconverted) sequence, and having a bisulfite converted sequence (e.g. Line-1 converted, Line-1 genomic)). The gBlocks artificial DNA fragments may further comprise as synthetic DNA standards (references) the expected sequences of the PCR products as amplified using the various primers for amplifying the bisulfite converted single-copy gene (e.g. short hTERT, long hTERT). Preferably, in a reference sample, the (unconverted and converted) repetitive DNA element templates are present in about equal amounts. Also, the single copy gene targets are preferably present in about equal amounts. Preferably, in a reference sample, the (unconverted and converted) repetitive DNA element templates and the single copy gene targets are mixed in a ratio that mimics or resembles the number of expected copies in a human bisulfite-converted DNA sample (e.g. 200:1 for LINE-1 vs. hTERT).

The short hTERT and long hTERT include the single-copy sequence based on the human reference genome.

The Line-1 converted and Line-1 genomic sequences as provided herein for the DNA references include a consensus repeat sequence based on the human reference genome.

The total amount of template DNA in a reference sample is preferably in the order of that expected in the test sample. A plurality of reference samples may be used with different amounts of template DNA for different reference samples. For instance, one may use 2 concentrations of template DNA in two different reference samples for preparing the standard curve. Alternatively, the standard curve may be used on a single template DNA concentration. Preferably, three, four, 5, 6, 7, 8, 9, or 10 reference samples are used, each having a different template DNA concentration. Very suitable concentration ranges for the template DNA in the reference samples include a range of from 0.1-100 ng/μl, or 0.5-50 ng/μl.

Hence, when using the four different gBlocks in a 50 ng/μl concentration, these are preferably mixed in ratios expected to be representative of their copy number the human genome (1:1:200:200 for short hTERT, long hTERT, LINE-1 converted and LINE-1 genomic, respectively).

The present invention provides an artificial ‘bisulfite treated DNA’ as a reference standard. The present invention allows for the determination of a global conversion efficiency of a bisulfite-treated DNA sample with the least primer pairs possible. For that purpose the present invention is aimed at using a repetitive DNA element as defined above, for instance, and preferably, Line-1. The means and methods of this invention target both the genomic and converted versions of the repetitive DNA element. In the case of Line-1 repeats, the human genome provides multiple copies of this gene with a very similar DNA sequence. However, some differences in the sequences may occur, such as single nucleotide differences. One of skill in the art will understand that consensus sequences may be obtained for these various sequences, and that based on such consensus sequences, appropriate primers and probes can be designed, comprising ambiguous bases if needed, to amplify and detect essentially all variants of the repetitive DNA element. When designing the reference DNA standards as explained herein, the skilled person will further appreciate that the various primers in a multiplex assay preferably have comparable melting temperatures.

The means and methods of this invention may be used in very diverse fields of study, such as health and disease, cancer, evolution, forensics, aging, developmental biology, cellular and molecular biology, clinical epigenetics, epidemiology, single-cell analysis, paediatrics, lifestyle and exercise research, plant biology, microbiology, stem cell biology etc.

### EXAMPLES

### Example 1. Materials and Methods

### DNA Samples

In this study, several DNA samples were used for both optimization and development of qBiCo. Synthetic double-stranded DNA fragments, five in number, gBlocks (Integrated DNA technologies, USA), were designed to have the expected PCR product sequences as generated by qPCR when using the proposed assays (Table 1). These five sequences were used to optimize and develop the method. Additionally, three DNA standards were used for assessing the performance of the 10 bisulfite conversion kits. The DNA standards for this experiment were Human Methylated DNA (250 ng/μl) (ZymoResearch, USA), Human high methylated genomic DNA (100 ng/μl) (EpigenDX, USA) and Quantifiler™ THP DNA Standard (100 ng/μl) (ThermoFisher, USA). For the validation of the qBiCo the Human high methylated genomic DNA (100 ng/μ1) (EpigenDX) standard was used while for the best ranked conversion kit the Human Methylated DNA (250 ng/μl) (ZymoResearch) standard was used. Finally, for assessing the specificity of the method, non-human DNA was included in the study. More specifically, extracted DNA derived from species Felis catus (cat), Canis lupus familiaris (dog), Mus musculus (mouse), Rattus (rat), Gallus gallus domesticus (chicken), Sus scrofa domesticus (pig) and Bos Taurus (cow) were tested for non-specific amplification. Human high methylated genomic DNA (100 ng/μl) (EpigenDX) was tested along with the mentioned species as a positive control.

TABLE 1
Primers and probes in qBiCo assay

| | | | | SEQ ID |
|---|---|---|---|---|
| Assay | | Sequence (5′→3′) | Label | NO |
| short | Forward Primer | TGGGTTTTAGAGTTGATTTTT | | SEQ ID |
| hTERT | (5′→3′) | | | NO: 1 |
| | Reverse Primer | ACCACATTAAACAATCCCCT | | SEQ ID |
| | (5′→3′) | | | NO: 2 |
| | Probe (5′→3′) | AATTAGGTATAGGGGATTTGGTTTTAGT | ZENTM | SEQ ID |
| | | | Quencher + | NO: 3 |
| | | | 5′FAMTM dye | |
| long | Forward Primer | AGGGTTTTAGTTAGAAGATT | | SEQ ID |
| hTERT | (5′→3′) | | | NO: 4 |
| | Reverse Primer | CATTATATATTAACTTATTCCCAC | | SEQ ID |
| | (5′→3′) | | | NO: 5 |
| | Probe (5′→3′) | GTGTTGGGGTTATTTTTTTGTATTTGG | ZENTM | SEQ ID |
| | | | Quencher + | NO: 6 |
| | | | 5′HEXTM dye | |
| Line-1 | Forward Primer | TTTTGAGTTAGGTGTGGGATATA | | SEQ ID |
| converted | (5′→3′) | | | NO: 7 |
| | Reverse Primer | AAAATCAAAAAATTCCCTTTC | | SEQ ID |
| | (5′-37) | | | NO: 8 |
| | Probe (5′→3′) | TGGGAGTGATTTGATTTTTTAGGTGY | 5′TEX 615 dye | SEQ ID |
| | | | | NO: 9 |
| Line-1 | Forward Primer | GAGCCAGGTGTGGGATATAA | | SEQ ID |
| genomic | (5′→3′) | | | NO: 10 |
| | Reverse Primer | TCAAAGAAAGGGGTGACAGA | | SEQ ID |
| | (5′→3′) | | | NO: 11 |
| | Probe (5′→3′) | TGGGAGTGACCCGATTTTCCAGGTG | 5′Cy5TM dye | SEQ ID |
| | | | | NO: 12 |
| IPC | Forward Primer | GGTGATTTTTAATAATTTTTGAGG | | SEQ ID |
| | (5′→3′) | | | NO: 13 |
| | Reverse Primer | ACTTTCAACCCATTACCTCAT | | SEQ ID |
| | (5′→3′) | | | NO: 14 |
| IPC | Probe (5′→3′) | AGTGTTCGTATAGGTTTTTTTTTTTAGT | 5′Cy5.5TM dye | SEQ ID |
| | | | | NO: 15 |

TABLE 2
gBlock amplicon sequences

| | | SEQ ID |
|---|---|---|
| Assay | gBlock (5′→3′) | NO: |
| short | TGGGTTTTAGAGTTGATTTTTGTGAATTAAATTAAAATTAGGTATAGGGG | SEQ ID |
| hTERT | ATTTGGTTTTAGTATAGGGGATTGTTTAATGTGGT | NO: 16 |
| long | AGGGTTTTAGTTAGAAGATTAGGGTTTTTTTAGTTTTTTTGTATATTCGAG | SEQ ID |
| hTERT | TTTTTGGGGGGTTTTGTGATATTTTATGTTTTAAATTAGGATGTTTGTAGA | NO: 17 |
| | GGGAGTTGGTAGTAGATTTCGTTAGAGGTAATATAGTTTTTGGGTTGGG | |
| | GATTTCGACGTGGTGTTGGGGTTATTTTTTTGTATTTGGGGGAGGGTTA | |
| | GGGTTTTTTTTGTGGGAATAAGTTAATATATAATG | |
| Line-1 | TTTTGAGTTAGGTGTGGGATATAATTTTTTGGTGTGTTGTTTTTTAAGTTT | SEQ ID |
| converted | TTTGGAAAAGTGTAGTATTTAGGGTGGGAGTGATTTGATTTTTTAGGTGT | NO: 18 |
| | TGTTTGTTATTTTTTTTTTTGATTAGGAAAGGGAATTTTTTGATTTT | |
| Line-1 | GAGCCAGGTGTGGGATATAATCTCCTGGTGTGCTGTTTTTTAAGCCCTT | SEQ ID |
| genomic | TGGAAAAGTGCAGTATTTAGGGTGGGAGTGACCCGATTTTCCAGGTGC | NO: 19 |
| | CGTCTGTCACCCCTTTCTTTGA | |
| IPC | GGTGATTTTTAATAATTTTTGAGGGAGTATTGGTATAGGCGATAGAGTGT | SEQ ID |
| | TCGTATAGGTTTTTTTTTTTAGTGGATGATGAGGTAATGGGTTGAAAGT | NO: 20 |

Forward and reverse primer binding sites are indicate (underlined), as well as probe annealing site (italics, underlined).

### Genomic DNA Quantitation/Quantification

All the DNA samples included in this study were quantified prior to bisulfite treatment to assess for their DNA quantity and quality. Quantifiler DUO DNA quantification kit (ThermoFisher, USA) was used for measuring the DNA quantity of the DNA samples. Quantifiler HP DNA quantification kit (ThermoFisher, USA) was used to assess both DNA quantity and quality of the DNA samples. Quantifiler HP (ThermoFisher) is an upgraded quantification kit which provides information not only for the DNA amount by targeting a small autosomal diploid sequence in the genome, but also for the fragmentation level of the DNA itself by targeting a large autosomal diploid sequence in the genome. The fraction of the small and the large fragment provides information concerning the fragmentation level of the DNA.

### Bisulfite Conversion

Bisulfite conversion is the gold standard procedure for DNA methylation analysis. Nowadays, there are many commercially available bisulfite conversion kits which are promising conversion efficiency >99%, DNA recovery >80% as well as DNA fragment range between 200 bp and 2000 bp in some cases. Part of the current study is to compare the performance of 10 different commercially available bisulfite conversion kits. The selected bisulfite conversion kits belong to different companies and are popular in the epigenetic field. The kits use similar protocols concerning their basic structure and steps (DNA conversion, DNA binding, desulphonation, washing and elution of DNA). However, there is variance within some steps, such as conversion incubation step in respect of incubation time and temperature of conversion (Table 3).
Moreover, there are different spin columns in every kit concerning the width and size of binding surface of the column. All kits are optimized to convert efficient DNA within the range of 100 ng-2 μg. All kits suggest that the optimal DNA amount to be treated is 200 ng.

TABLE 3
Variance in conversion incubation step across
the 10 selected bisulfite conversion kits

| Kits | Conversion incubation |
|---|---|
| Promega | 8 min at 98° C./60 min at 54° C. |
| ThermoFisher | 10 min at 98° C./150 min at 60° C. |
| ZymoResearch | 14-16 hours at 50° C. |
| Qiagen | 5 min at 95° C./25 min at 60° C./5 min at 95° C./ |
| | 85 min at 60° C./5 min at 95° C./175 min at 60° C. |
| Diagenode | 8 min at 98° C./60 min at 54° C. |
| Epigentek | 90 min 65° C. |
| Active motif | 30 sec at 95° C./20 min at 58° C. |
| ABCam | 20 min at 95° C. |
| Sigma-Aldrich | 90 min 65° C. |
| analytikjena | 45min at 85° C. |

MethylEdge® Bisulfite Conversion System (Promega, USA); EpiJET Bisulfite Conversion Kit (ThermoFisher, USA); EZ DNA Methylation Kit (Zymo Research, USA); EpiTect Bisulfite Kit (Qiagen, USA); Premium Bisulfite Kit (Diagenode, USA); Methylamp DNA Modification Kit Cat #P-1001 (Epigentek, Farmingdale, N.Y., USA); Bisulfite conversion kit, Catalog No. 55016 (Active Motif, Carlsbad, Ca, USA); Fast Bisulfite Conversion Kit (ab117127) (Abcam, USA); Imprint® DNA Modification Kit (Sigma Aldrich/Merck, USA); innuCONVERT Bisulfite Basic Kit (Analytik Jena AG, DE)
For evaluating the performance of the selected bisulfite conversion kits, three DNA standards (Human Methylated DNA (250 ng/μl) (ZymoResearch), Human high methylated genomic DNA (100 ng/μl) (EpigenDX) and Quantifiler™ THP DNA Standard (100 ng/μl) (ThermoFisher)) underwent bisulfite conversion in five different DNA amount. More specifically, 200 ng, 100 ng, 50 ng, 10 ng, 1 ng from each sample underwent bisulfite conversion. The specific DNA input was selected to test a) each kit for the optimal amount b) the limits of the performance of each kit. In the second part of the current study, the Human Methylated DNA (250 ng/μl) (ZymoResearch) standard was used to test the performance reproducibility of the best in ranking bisulfite conversion kit, EZ DNA Methylation Kit (ZymoResearch, USA). For this purpose, a wider range of DNA amounts was used as input for bisulfite conversion. The DNA amounts that underwent bisulfite conversion were 200 ng, 100 ng, 50 ng, 25 ng, 12.5 ng, 6.25 ng, 3.125 ng and 1.563 ng. The bisulfite conversion was repeated in a different time point to assess the performance reproducibility of the kit. Finally, 200 ng of DNA standard Human high methylated genomic DNA (100 ng/μl) (EpigenDX) were treated with sodium bisulfite and used for the validation study of qBiCo. The best ranked bisulfite conversion kit, EZ DNA Methylation Kit (ZymoResearch) was employed for treating the sample.

### Bisulfite-Converted DNA Fragmentation Analysis

To assess the performance of qBiCo in comparison with existing methods that can be used for bisulfite-converted DNA fragmentation analysis, the samples were analyzed with bioanalyzer technology (Agilent Technologies, USA). For this purpose the two out of the eight bisulfite converted samples for each of the two conversion experiments prepared for assessing the performance of the best ranked kit, EZ DNA Methylation Kit (ZymoResearch), were used. Particularly, the converted samples with DNA input 200 ng, as the optimal tested amount, was tested for fragmentation using the bioanalyzer technology. Additionally, 12.5 ng of DNA input for conversion was tested along with the 200 ng for assessing how the conversion performs for lower DNA qualities. As bisulfite converted DNA is mostly single-stranded, the fragmentation was assessed by using Agilent RNA 6000 Pico Kit (Agilent Technologies, USA) and the bioanalyzer instrument (Agilent Technologies, USA). The four samples were run in duplicate for both DNA bisulfite conversions.

### Bisulfite-Converted DNA Quantification

qBiCo was additionally evaluated for the quantification performance in comparison with available and common used methods. For this purpose, the same four bisulfite converted samples which were used for fragmentation assessment with bioanalyzer, were analyzed by using spectrophotometric and fluoromentric quantification. Particularly, we used Nanodrop spectrophotometer (ThermoFisher) on ssDNA mode and Qubit fluorometer (ThermoFisher) with ssDNA assay kit for measuring the bisulfite-converted DNA quantity. All samples were measured in duplicate.
qBico Assay Design
For qBiCo development five primer sets each comprising one probe were designed (Table 1). Each probe carries a different fluorophore to provide a signal which can be separated from different probes according to different dyes. Two out of five assays are designed to target the human L1 repetitive element in genomic DNA (119 pb; Line-1 genomic SEQ ID NO:19) and bisulfite converted DNA (148 bp; Line-1 converted, SEQ ID NO:18). The two corresponding probes for these primer sets were designed to distinguish between converted and genomic DNA and were labelled with TEX616™ and Cy5™ respectively. These two assays were designed to provide information about the conversion efficiency as the L1 element covers 17% of the entire genome. Using bioinformatics tools, the primers/probes were designed in a way to maximize the positions that they bind, which correspond to ˜200 in total.
For determining bisulfite conversion efficiency, two primer and probe sets, labelled with different dyes, were designed to target genomic and converted long interspersed element 1 (L1/LINE-1).

Additionally, two assays designed to target the bisulfite-converted version of a long (235 bp; long hTERT) and a short (85 bp; short hTERT) fragment both belonging to human telomerase reverse transcriptase gene (hTERT). These assays were included for assessing the quality of the converted DNA by addressing the degradation level of DNA. Moreover, the short fragment (hTERT) was used for assessing the quantity of bisulfite-converted DNA. The probes designed to hybridize to long and short fragments of the hTERT gene were labelled with FAM™ and HEX™ fluorophores respectively. Finally, a set of primers was designed to amplify a synthetic non-human DNA sequence (99 bp; IPC). The corresponding probe for this assay was labelled with Cy5.5™ and used as an internal positive control (IPC) as well as means for assessing PCR inhibition (Table 1).

TABLE 4
Specification of five assays included in qBiCo.

| | | Amplicon | | |
|---|---|---|---|---|
| | Genomic | length | Copy | |
| Target | Location | (bp) | Number | Dye |
| Human Target, | hTERT | 85 | single-copy | HEX ™ |
| short autosomal | | | | |
| Human Target, | hTERT | 235 | single-copy | FAM ™ |
| long autosomal | | | | |
| Human Target, | Line-1 | 148 | multicopy | TEX616 ™ |
| converted autosomal | | | | |
| Human Target, | Line-1 | 119 | multicopy | Cy5 ™ |
| genomic autosomal | | | | |
| Internal | NA | 99 | Synthetic | Cy5.5 ™ |
| PCR Control | | | template | |

Genome browser Ensembl was used to locate the region of interest in the human genome (GRCh37) and extract the surrounding DNA sequence. The expected bisulfite-converted DNA sequences were obtained by the MethylPrimer software and were used to design bisulfite specific primers and probes. The sets of primers and probes designed in silico by using Bisearch software. Additionally, five synthetic double stranded DNA fragments, gBlocks (Integrated DNA technologies, IDT), were designed to mimic each of the expected PCR products derived from the assays (Table 2). These fragments were used as DNA samples for optimizing and validating the method and preferred over human DNA as the copy number of each fragment per reaction is known. The four different gBlocks (except for internal positive control) were mixed in ratios expected to be representative concerning the same copy number as in 50 ng of human DNA. Serial dilutions by a factor two were made using the mixture of gBlocks (50 ng/μl) and resulted in eight standards with concentration range 50-0.39 ng/μl. The IPC input was the same in all reaction thus was not added in the mixture of gBlocks.
Quantitative (q) PCR—qBico
The five assays specified above were amplified simultaneously in a PCR reaction using the primer and probe pairs described above. The PCR reaction was optimized in final volume 20 μl, containing, 10 μl of 2× EpiTect® MethyLight qPCR (Qiagen, Germany) reagent, 2 μl of 25 mM MgCl₂ (Applied Biosystems, USA), 0.8 μl of BSA (BioLabs, USA), 3.2 μl of nuclease-free water, 2 μl of primer/probe mix, 1 μl of internal positive control gBlock and 1 μl of bisulfite-converted DNA template. The primer/probe mix was optimized according to Table 5. The cycling conditions for this qPCR consisted of polymerase activation and denaturation (95° C., 5 min), 33 cycles of denaturation (95° C., 15 s), annealing (56° C., 30 s) and extension (60° C., 70 s). The optimized assays were tested for their performance such as efficiency, reproducibility, repeatability, specificity, sensitivity and robustness. In all experiments a serial dilution of eight standards were prepared from 50 ng/μl gBlocks mix in nuclease-free water to obtain concentrations in a range from 50-0.39 ng/μl. Additionally, the qPCR amplifications were performed in triplicate to assess the repeatability of the assays. As a negative control nuclease-free water was used. All reactions were performed in a 96-well plate (Bio-Rad, USA) in a qPCR instrument Bio-rad CFX96 Touch™ Real-Time PCR Detection system (Bio-Rad).

TABLE 5
Concentration combination of
primer/probe master mix of qBiCo.

| | | Concentration |
|---|---|---|
| Assay | Oligos | (μM) |
| short hTERT | Forward Primer | 0.6 |
| | Reverse Primer | 0.6 |
| | Probe | 0.3 |
| long hTERT | Forward Primer | 0.2 |
| | Reverse Primer | 0.2 |
| | Probe | 0.1 |
| Line-1 | Forward Primer | 0.1 |
| converted | Reverse Primer | 0.1 |
| | Probe | 0.1 |
| Line-1 | Forward Primer | 0.1 |
| genomic | Reverse Primer | 0.1 |
| | Probe | 0.1 |
| IRC | Forward Primer | 0.2 |
| | Reverse Primer | 0.2 |
| | Probe | 0.1 |

### Data Analysis

For assessing the efficiency of each assay, gBlock mix stock 50 ng/μl was used for making serial dilutions by factor of two, resulting in eight DNA standards with range 50-0.39 ng/μl. These dilutions were run in duplicate to obtain a standard curve for quantification of bisulfite-converted DNA. The equation of the standard curves were calculated by the instrument: C_{T}=m[log (Qty)]+b, where m is the slope, b the y-intercept and Qty, the starting DNA quantity of the each individual standard. The qPCR instrument also obtained information about the R², slope and efficiency. An R²≥0.99 indicated that the measured C_{T} values for the standard curves are close to the calculated C_{T} values for the regression line, but values >0,985 are also acceptable. The slope also indicated the PCR efficiency (E) and should be −3,6≥slope≥-3,3, corresponding to a 90-100% efficiency. The bisulfite DNA recovery was directly measured by the amount of the hTERT short autosomal fragment. Furthermore, the conversion efficiency rate was calculated according to the following formula: Conversion efficiency=[amount of L1 converted (ng)]/[amount of L1 converted (ng)+amount of genomic L1 (ng)]*100%. Similar, the fragmentation index and the presence of PCR inhibitor were calculated as follows: Fragmentation index=Fragmentation degree [HP]− [amount of hTERT long (ng)]/[amount of hTERT small (ng)]. Lastly, as for the assessment of PCR inhibitors, an IPC Ct threshold was determined as follows: Inhibition presence=average of IPC CT in sample >(average+standard deviation of IPC Cq value based on the DNA standards).

1. A method to determine bisulfite conversion efficiency following bisulfite conversion of unmethylated cytosine to uracil in a genomic DNA sample, the method comprising the steps of:
a) providing a sample of bisulfite-treated genomic DNA, said genomic DNA comprising a multi-copy target DNA sequence that is a repetitive DNA element, and wherein said bisulfite treatment converts unmethylated cytosine in the sequence of said repetitive DNA element to uracil to thereby generate bisulfite-converted copies of said repetitive DNA element;
b) providing a first set of amplification primers for amplifying by qPCR bisulfite-converted copies of said repetitive DNA element present in said bisulfite-treated genomic DNA sample and for generating a first amplicon, and providing a first detection probe labeled with a first detectable label for detecting said first amplicon by qPCR;
c) providing a second set of amplification primers for amplifying by qPCR unconverted copies of said repetitive DNA element present in said bisulfite-treated genomic DNA and for generating a second amplicon, and providing a second detection probe labeled with a second detectable label for detecting said second amplicon by qPCR;
d) performing a multiplex qPCR with said first and second set of amplification primers and probes and using said bisulfite-treated genomic DNA sample as a PCR template to generate said first amplicon with said first primer set from converted copies of said repetitive DNA element present in said bisulfite-treated genomic DNA sample, and to generate said second amplicon with said second primer set from unconverted copies of said repetitive DNA element present in said bisulfite-treated genomic DNA sample;
e) providing a first qPCR standard curve for determining the amount of bisulfite-converted copies of said repetitive DNA element present in said bisulfite-treated genomic DNA sample, and providing a second qPCR standard curve for determining the amount of unconverted copies of said repetitive DNA element present in said bisulfite-treated genomic DNA sample, wherein said first and second standard curves are obtained by performing a multiplex qPCR on a reference sample comprising as a PCR template a known amount of (i) a first synthetic DNA standard consisting of an oligonucleotide comprising a probe binding side complementary to said first detection probe flanked by primer binding sides complementary to said first set of amplification primers, and (ii) a second synthetic DNA standard consisting of an oligonucleotide comprising a probe binding side complementary to said second detection probe flanked by primer binding sides complementary to said second set of amplification primers;
f) determining the amount or concentration of converted and unconverted copies of said repetitive DNA element in said bisulfite-treated genomic DNA sample based on the generation of said first and second amplicon, respectively in step d), and based on said first and second qPCR standard curve provided in step e);
g) calculating the bisulfite conversion efficiency of the bisulfite treatment based on the ratio between the amount or concentrations of converted and unconverted copies of said repetitive DNA element present in said bisulfite-treated genomic DNA sample determined in step f).

2. The method according to claim 1, wherein said repetitive DNA element is a long interspersed nuclear element (LINE), an L1 repetitive element (LINE1).

3. The method according to claim 1, wherein said first set of amplification primers comprises a forward primer comprising the nucleotide sequence of SEQ ID NO:7 and a reverse primer comprising the nucleotide sequence of SEQ ID NO:8; and optionally wherein the first detection probe comprises the nucleotide sequence of SEQ ID NO:9.

4. The method according to claim 1, wherein said second set of amplification primers comprises a forward primer comprising the nucleotide sequence of SEQ ID NO:10 and a reverse primer comprising the nucleotide sequence of SEQ ID NO:11; and optionally wherein the second detection probe comprises the nucleotide sequence of SEQ ID NO:12.

5. The method according to claim 1, wherein said first synthetic DNA standard is the sequence of SEQ ID NO:18 and wherein said second synthetic DNA standard is the sequence of SEQ ID NO:19.

6. The method according to claim 1, wherein the bisulfite conversion efficiency is calculated using the formula: ([amount of first amplicon (ng)]/[amount of first amplicon (ng)+amount of second amplicon (ng)])×100%.

7. The method according to claim 1, wherein said method is a method for simultaneously determining a bisulfite conversion efficiency, a bisulfite converted DNA quantity and a level of DNA fragmentation following bisulfite conversion; wherein said bisulfite-treated genomic DNA sample further comprises a single-copy gene sequence, said method further comprising the steps of:
h) providing a third set of amplification primers for amplifying by qPCR at least a part of said single-copy gene sequence that is bisulfite-converted and for generating a third amplicon; and a third detection probe labeled with a third detectable label for detecting said third amplicon by qPCR;
i) providing a fourth set of amplification primers for amplifying by qPCR at least a part of said single-copy gene sequence that is bisulfite-converted and for generating a fourth amplicon, and providing a fourth detection probe labeled with a fourth detectable label for detecting said fourth amplicon by qPCR, wherein the third and fourth set of amplification primers are designed such that the length (in bp) of said fourth amplicon is longer than the length (in bp) of said third amplicon;
j) performing the multiplex qPCR of step d) which further includes said third and fourth set of primers and probes to thereby further generate said third amplicon with said third primer set and said fourth amplicon with said fourth primer set;
k) providing a third qPCR standard curve for determining the amount of converted and optionally fragmented copies of said single copy gene sequence present in said bisulfite-treated genomic DNA sample, and providing a fourth qPCR standard curve for determining the amount of converted and non-fragmented copies of said single-copy gene sequence present in said bisulfite-treated genomic DNA sample, wherein said third and fourth standard curves are obtained by performing a multiplex qPCR on a reference sample comprising as a PCR template a known amount of (iii) a third synthetic DNA standard consisting of an oligonucleotide comprising a probe binding side complementary to said third detection probe flanked by primer binding sides complementary to said third set of amplification primers, and (iv) a fourth synthetic DNA standard consisting of an oligonucleotide comprising a probe binding side complementary to said fourth detection probe flanked by primer binding sides complementary to said fourth set of amplification primers;
l) determining the amount or concentration of converted and optionally fragmented copies of said single copy gene sequence present in said bisulfite-treated genomic DNA sample based on the generation of said third amplicon in step j), and based on said third qPCR standard curve provided in step k), to thereby provide a bisulfite-converted DNA quantity in said bisulfite-treated genomic DNA sample,
m) determining the amount or concentration of converted and non-fragmented copies of said single copy gene sequence present in said bisulfite-treated genomic DNA sample based on the generation of said fourth amplicon in step j), and based on said fourth qPCR standard curve provided in step k);
n) and calculating the level of DNA fragmentation following bisulfite conversion by comparing the amount of said converted and optionally fragmented copies with the amount of converted and non-fragmented copies present in said bisulfite-treated genomic DNA sample as determined in steps 1) and m).

8. The method according to claim 7, wherein the third amplicon is 60-100 bps, about 85 bps, and said fourth amplicon is 150-350 bps, about 235 bps.

9. The method according to claim 7, wherein said single copy gene sequence is a (human) telomerase reverse transcriptase gene (TERT).

10. The method according to claim 7, wherein said third set of amplification primers comprises a forward primer comprising the nucleotide sequence of SEQ ID NO:1 and a reverse primer comprising the nucleotide sequence of SEQ ID NO:2; and optionally wherein the third detection probe comprises the nucleotide sequence of SEQ ID NO:3.

11. The method according to claim 7, wherein said fourth set of amplification primers comprises a forward primer comprising the nucleotide sequence of SEQ ID NO:4 and a reverse primer comprising the nucleotide sequence of SEQ ID NO:5; and optionally wherein the fourth detection probe comprises the nucleotide sequence of SEQ ID NO:6.

12. The method according to claim 7, wherein said third synthetic DNA standard is the sequence of SEQ ID NO:16 and wherein said fourth synthetic DNA standard is the sequence of SEQ ID NO:17.

13. The method according to claim 7, wherein the amount of said first and second synthetic DNA standard relative to the amount of said third and fourth synthetic DNA standard in said reference sample reflect the ratio of copy numbers of said repetitive DNA element and said single-copy gene sequence in genomic DNA in said bisulfite-treated genomic DNA sample, wherein the ratio between said first and second synthetic DNA standard is about 1 and wherein the ratio between said third and fourth synthetic DNA standard is about 1, wherein the ratio between the copy number of said first, second third and fourth synthetic DNA standard in said reference sample is 200:200:1:1, respectively.

14. A method for simultaneously determining a bisulfite conversion efficiency, a bisulfite converted DNA quantity, a level of DNA fragmentation following bisulfite conversion, and PCR inhibition, said method comprising the steps of:
performing a method claim 1; and further comprising the steps of:
adding to said bisulfite-treated genomic DNA sample an artificial DNA sequence in a known amount, wherein the artificial DNA sequence comprises the nucleotide sequence of SEQ ID NO:20;
providing a fifth set of amplification primers for amplifying by qPCR said artificial DNA sequence and for generating a fifth amplicon; and a fifth detection probe labeled with a fifth detectable label for detecting said fifth amplicon by qPCR;
performing said multiplex qPCR which further includes said fifth set of primers and probe to thereby further generate said fifth amplicon with said fifth primer set;
determining the amount of said fifth amplicon, on the basis of qPCR Cq values, and determine the level of PCR inhibition by comparing the determined amount of said fifth amplicon with the amount of artificial DNA sequence added to said bisulfite-treated genomic DNA sample.

15. The method according to claim 14, wherein said fifth set of amplification primers comprises a forward primer comprising the nucleotide sequence of SEQ ID NO:13 and a reverse primer comprising the nucleotide sequence of SEQ ID NO:14; and optionally wherein the fourth detection probe comprises the nucleotide sequence of SEQ ID NO:15.

16. A qPCR kit comprising
a first set of amplification primers for amplifying bisulfite converted copies of a genomic multi-copy target DNA sequence that is a repetitive DNA element and for generating a first amplicon;
a first detection probe labeled with a first detectable label for detecting said first amplicon;
a second set of amplification primers for amplifying unconverted copies of said repetitive DNA element by qPCR and for generating a second amplicon;
a second detection probe labeled with a second detectable label for detecting said second amplicon;
wherein said qPCR kit optionally further comprises
a third set of amplification primers for amplifying a part of a genomic single copy gene sequence that is bisulfite converted and for generating a third amplicon;
a third detection probe labeled with a third detectable label for detecting said third amplicon;
a fourth set of amplification primers for amplifying a part of said genomic single copy gene sequence that is bisulfite converted and for generating a fourth amplicon that is longer in length than said third amplicon;
a fourth detection probe labeled with a fourth detectable label for detecting said fourth amplicon; and/or
a fifth set of amplification primers for amplifying an internal positive control DNA sequence and for generating a fifth amplicon, wherein said fifth set of amplification primers comprises a forward primer comprising the nucleotide sequence of SEQ ID NO:13 and a reverse primer comprising the nucleotide sequence of SEQ ID NO:14;
a fifth detection probe labeled with a fifth detectable label for detecting said fifth amplicon by qPCR, wherein said fifth detection probe comprises the nucleotide sequence of SEQ ID NO:15;
wherein said qPCR kit optionally further comprises
a first synthetic DNA standard consisting of the nucleotide sequence of said first amplicon, wherein said first synthetic DNA standard is the sequence of SEQ ID NO:18;
a second synthetic DNA standard consisting of the nucleotide sequence of said second amplicon, wherein said second synthetic DNA standard is the sequence of SEQ ID NO:19; and/or;
a third synthetic DNA standard consisting of the nucleotide sequence of said third amplicon, wherein said third synthetic DNA standard is the sequence of SEQ ID NO:16; and/or
a fourth synthetic DNA standard consisting of the nucleotide sequence of said fourth amplicon, wherein said fourth synthetic DNA standard is the sequence of SEQ ID NO:17; and/or
an internal positive control DNA standard, consisting of the sequence of SEQ ID NO:20.

17. A qPCR kit according to claim 16, comprising, in combination, the primers and probes of SEQ ID NO:7-9 adapted to support a 5plex PCR assay, the primers and probes of SEQ ID NO:1-15 adapted to support a 5plex PCR assay and/or synthetic DNA standards of SEQ ID NOs:16-19, said kit optionally further comprising the internal positive control comprising the DNA sequence of SEQ ID NO:20.

18. The method according to claim 14, wherein the first, second, third, fourth and fifth detectable label are different.

19. A method for measuring bisulfite conversion efficiency in a bisulfite treated genomic DNA sample; wherein a first synthetic oligonucleotide has a nucleotide sequence that corresponds to the sequence of a bisulfite converted copy of a repetitive genomic DNA element and a second synthetic oligonucleotide has a nucleotide sequence that corresponds to the sequence of a non-bisulfite converted copy of said repetitive genomic DNA element, wherein the first synthetic oligonucleotide is SEQ ID NO: 18 or 19 and the second synthetic oligonucleotide is SEQ ID NO: 16 or 17.
